(19) United States
(12) Patent Application Publication
(10) Pub. No.: US 2014/0004499 A1
(43) Pub. Date: Jan. 2, 2014

(54) BIOSURFACE ENGINEERING

(71) Applicants: Nicolai Vladimirovich Bovin, Moscow (RU); Andrey Alfredovich Formanovsky, Moscow (RU); Stephen Micheal Henry, Auckland (NZ); Stephen Robert Parker, Auckland (NZ); Inna Stanislavovna Popova, Moscow (RU)

(72) Inventors: Nicolai Vladimirovich Bovin, Moscow (RU); Andrey Alfredovich Formanovsky, Moscow (RU); Stephen Micheal Henry, Auckland (NZ); Stephen Robert Parker, Auckland (NZ); Inna Stanislavovna Popova, Moscow (RU)

(21) Appl. No.: 13/978,691

(22) PCT Filed: Jan. 6, 2012
(86) PCT No.: PCT/NZ2012/000001

§ 371 Date: Sep. 19, 2013

(30) Foreign Application Priority Data
Jan. 6, 2011 (NZ) 590342
Nov. 1, 2011 (NZ) 596147

(51) Int. Cl. C07K 7/02; C07J 41/00

(52) U.S. Cl. CPC C07K 7/02; C07J 41/0055
USPC 435/2; 536/5; 530/323

(57) ABSTRACT

The invention relates to methods of localizing biofunctional moieties (F) to surfaces and synthetic constructs of the general structure F-S-S′ for use in such methods. F is the biofunctional moiety, S is a spacer covalently linking F to S′, and S′ is sterol. In particular, the invention relates to the preparation of biofunctional surfaces and surface modified biological structures including cells (kodecytes), enveloped viruses (kodevirions) and liposomes or virosomes (kodesomes).

### FIELD OF INVENTION

The invention relates to methods of localising biofunctional moieties (F) to surfaces and synthetic constructs of the general structure F-S-S′ (where S′ is a sterol) for use in such methods. In particular, the invention relates to the preparation of biofunctional surfaces and surface modified biological structures including cells (kodecytes), enveloped viruses (kodevirions) and liposomes or virosomes (kodesomes).

### BACKGROUND ART

The ability to localise biofunctional moieties to solid surfaces and membranes has utility in both diagnostic and therapeutic applications. For example, immunosorbent based applications are dependent on the use of an insoluble preparation of an antigen (the biofunctional moiety) to bind specific antibodies from a mixture. Similarly, the publication of Spedden (2008) describes the identification and immobilization of glycosylated molecules having biomimetic properties on solid state surfaces and films or on membranes arising at the interface between non-polar and polar materials. The biomimetic glycosylated films and particles constructed therefrom are identified as having industrial, environmental, diagnostic and/or therapeutic utility in the binding, capture, and/or extraction of pathogens, toxins and/or contaminants, in vivo, in vitro or in situ.

The publication of Bovin et al (2005) describes the preparation of synthetic molecules that spontaneously and stably incorporate into lipid bilayers, including cell membranes. The synthetic molecules are used to effect qualitative and quantitative changes in the expression of cell surface antigens. Similarly, the publication of Korchagina et al (2008) describes the preparation and use of fluorescent cell markers of the structure F-S₁-S₂-L where F is a fluorophore, S₁-S₂ is a spacer linking F to L, and L is a diacyl lipid. The markers are used to localise the fluorophore (the biofunctional moiety) to the surface of cells and multi-cellular structures. In addition, the publications of Weinberg et al (2009) and Bovin et al (2009) describe the preparation of functional lipid constructs, including peptide-lipid constructs that are readily dispersible in aqueous media and have utility in diagnostic and therapeutic applications, including serodiagnosis.

It is an object of this invention to provide an alternative method of modifying surfaces by the localisation of biofunctional moieties. It is an object of this invention to provide an improved method of localizing functional moieties to artificial membranes, including liposomes. These objects are to be read disjunctively with the object of the invention to at least provide the public with a useful choice.

### STATEMENT OF INVENTION

In a first aspect the invention provides a method of localizing a hydrophilic biofunctional moiety to a surface comprising the step of contacting the surface with an aqueous solution of a construct of the structure F-S-S′ where F is the biofunctional moiety, S is a spacer covalently linking F to S′, and S′ is sterol. S is selected to provide a construct that forms a stable, monophasic dispersion in water in the absence of detergents or organic solvents (water dispersible).

Preferably, F is selected from the group consisting of: biotin and O-linked oligosaccharides. Preferably, when the biofunctional moiety (F) is an oligosaccharide, the biofunctional moiety (F) is selected from the group consisting of: GalNAcα3(Fucα2)Galβ-; Galα3(Fucα2)Galβ-; GalNa₃(Fucα2)Galβ-; Fucα2Galβ-; Galβ4GlcNAcβ3(Galβ4GlcNAcβ6)Galβ-; Galβ4GlcNAcβ3-; Galβ4Glcβ-; Galβ3GlcNAcβ-; Galβ3(Fucα4)GlcNA4-; Fucα2Galβ3(Fucα4)GlcNAcβ-; GalNAcα3(Fucα2)Galβ3(Fucα4)GlcNAcβ-Galα3(Fucα2)Galβ3(Fucα4)GlcNAcβ-; Galβ4(Fucα3)GlcNAcβ-; Fucα2Galβ4(Fucα3)GlcNAcβ-; NeuAcα2-3 Galβ3(Fucα4)GlcNA4-; NeuAcα2-3Galβ4(Fucα3)GlcNA0-; GalNAcβ4(NeuAcα2-3)Galβ4-; Galβ3GalNAcα-; NeuAcα2-3Galβ4-; NeuAcα2-6Galβ4-; Galα4Galβ4-; GalNAcβ3Galα4Galβ4-; Galα4Galβ4GlcNAcβ3-; Galβ3GalNAcβ3Galα4-; NeuAcα2-3Galβ3GalNAcβ3Galα4-; Galα3Galβ-; GalNAcα3GalNAcβ3Galα4-; GalNAcβ3GalNAcβ3Galα4-; Galβ1-4GlcNAc; Galβ1-3GlcNAc; SAα2-6Galβ1-4Glc; SAα2-3Galβ1-4Glc; SAα2-6Galβ1-4GlcNAc; SAα2-3Galβ1-4GlcNAc; SAα2-3Galβ1-3GlcNAc; Galβ1-4(Fucα1-3)GlcNAc; Galβ1-3(Fucα1-3)GlcNAc; SAα2-3Galβ1-3(Fucα1-4)GlcNAc; SAα2-3Galβ1-4(Fucal-3)GlcNAc; Galβ1-4GlcNAcβ1-4GlcNAc; Galβ1-3GlcNAcβ1-4GlcNAc; SAα2-6Galβ1-4GlcNAcβ1-4GlcNAc; SAα2-3Galβ1-4GlcNAcβ1-4GlcNAc; SAα2-3Galβ1-3GlcNA41-4GlcNAc; Galβ1-4(Fucα1-3)GlcNAcβ1-4GlcNAc; Galβ1-3(Fucα1-4)GlcNAcβ1-4GlcNAc; SAα2-3Galβ1-3(Fucα1-4)GlcNAcβ1-4GlcNAc; SAα2-3Galβ1-4(Fucα1-3)GlcNA41-4GlcNAc; SAα2-3Galβ1-3(Fucα1-4)GlcNAcβ1-4Gal; SAα2-3Galβ1-4(Fucα1-3)GlcNAβ1-4Gal; SAα2-3Galβ1-4GlcNAβ1-3Galβ1-4(Fucα1-3)GlcNAc; SAα2-6Galβ1-4GlcNAcβ1-3Galβ1-4(Fucα1-3)GlcNAc; SAα2-3Galβ1-4(Fucα1-3)GlcNAcβ1-3Galβ1-4Glc; SAα2-6Galβ1-4(Fucα1-3)GlcNAcβ1-3Galβ1-4Glc; SAα2-3Galβ1-4GlcNAcβ1-3Galβ1-4(Fucα1-3)GlcNAcβ1-3Galβ1-4Glc; SAα2-6Galβ1-4GlcNAcβ1-3Galβ1-4(Fucα1-3)GlcNAcβ1-3Galβ1-4Glc; SAα2-3Galβ1-4(Fucal-3)GlcNAcβ1-3Galβ1-4(Fucα1-3)GlcNAcβ1-3Galβ1-4Glc; SAα2-6Galβ1-4(Fucal-3)GlcNAcβ1-3Galβ1-4(Fucα1-3)GlcNAcβ1-3Galβ1-4Glc; SAα2-3Galβ1-4(Fucal-3)GlcNAcβ1-3Galβ1-4(Fucα1-3)Glc; SAα2-6Galβ1-4(Fucα1-3)GlcNAcβ1-3Galβ1-4(Fucα1-3)Glc; SAα2-3Galβ1-4GlcNAcβ1-3Galβ1-4(Fucα1-3)GlcNAcβ1-3Galβ1-4(Fucα1-3)Glc; SAα2-6Galβ1-4GlcNAcβ1-3Galβ1-4(Fucα1-3)GlcNAcβ1-3Galβ1-4(Fucα1-3)Glc; SAα2-3Galβ1-4(Fucα1-3)GlcNAcβ1-3Galβ1-4(Fucα1-3)GlcNAcβ1-3Galβ1-4(Fucα1-3)Glc; SAα2-6Galβ1-4(Fucα1-3)GlcNAcβ1-3Galβ1-4(Fucal-3)GlcNAcβ1-3Galβ1-4(Fucα1-3)Glc; SAα2-3Galβ1-3(Fucα1-4)GlcNAc; SAα2-6Galβ1-3(Fucα1-4(GlcNAc; SAα2-3Galβ1-3GlcNAcβ1-4Galβ1-4(Fucα1-3)GlcNAc; SAα2-6Galβ1-3GlcNAcβ1-4Galβ1-4(Fucα1-3)GlcNAc; SAα2-3Galβ1-3(Fucal-4)GlcNAcβ1-3Galβ1-4(Fucα1-3)GlcNAc; SAα2-6Galβ1-3(Fucα1-4)GlcNAcβ1-3Galβ1-4(Fucα1-3)GlcNAc; SAα2-3Galβ1-3(Fucα1-4)GlcNAcβ1-3Galβ1-4Glc; SAα2-6Galβ1-3(Fucα1-4)GlcNAcβ1-3Galβ1-4Glc; SAα2-3Galβ1-3GlcNAβ1-4Galβ1-4(Fucα1-3)GlcNAcβ1-3Galβ1-4Glc; SAα2-6Galβ1-3GlcNAβ1-4Galβ1-4(Fucα1-3)GlcNAβ1-3Galβ1-4Glc; SAβ2-3Galβ1-3(Fucα1-4)GlcNAcβ1-3Galβ1-4(Fucα1-3)GlcNAcβ1-3Galβ1-4Glc; SAα2-6Galβ1-3(Fucα1-4)GlcNAcβ1-3Galβ1-4(Fucα1-3)GlcNAcβ1-3Galβ1-4Glc; SAα2-3Galβ1-3(Fucα1-4)GlcNAcβ1-3Galβ1-4(Fucα1-3)Glc; SAα2-6Galβ1-3(Fucα1-4)GlcNAcβ1-3Galβ1-4(Fucα1-3)Glc; SAα2-3Galβ1-3GlcNAcβ1-4Galβ1-4(Fucα1-3)GlcNAcβ1-3Galβ1-4(Fucα1-3)Glc; SAα2-6Galβ1-3GlcNAcβ1-4Galβ1-4(Fucα1-3)GlcNAcβ1-3Galβ1-4(Fucα1-3)Glc; SAα2-3Galβ1-3(Fucα1-4)GlcNAcβ1-3Galβ1-4(Fucα1-3)GlcNAcβ1-3Galβ1-4(Fucα1-3)Glc; SAα2-6Galβ1-3(Fucα1-4)GlcNAcβ1-3Galβ1-4(Fucα1-3)GlcNAcβ1-3Galβ1-4(Fucα1-3)Glc; SAα2-3Galβ1-4GlcNAcβ1-3Galβ1-3(Fucα1-4)GlcNAc; SAα2-6Galβ1-4GlcNAcβ1-3Galβ1-3(Fucα1-4)GlcNAc; SAα2-3Galβ1-4(Fucα1-3)GlcNAcβ1-3Galβ1-3(Fucα1-4)GlcNAc; SAα2-6Galβ1-4(Fucα1-3)GlcNAcβ1-3Galβ1-3(Fucα1-4)GlcNAc; SAα2-3Galβ1-4GlcNAcβ1-3Galβ1-3(Fucα1-4)GlcNAcβ1-3Galβ1-4Glc; SAα2-6Galβ1-4GlcNAcβ1-3Galβ1-3(Fucα1-4)GlcNAcβ1-3Galβ1-4Glc; SAα2-3Galβ1-4(Fucα1-3)GlcNAcβ1-3Galβ1-3(Fucα1-4)GlcNAcβ1-3Galβ1-4Glc; SAα2-6Galβ1-4(Fucα1-3)GlcNAcβ1-3Galβ1-3(Fucα1-4)GlcNAcβ1-3Galβ1-4Glc; SAα2-3Galβ1-4(Fucα1-3)GlcNAcβ1-3Galβ1-3(Fucα1-4)Glc; SAα2-6Galβ1-4(Fucα1-3)GlcNAcβ1-3Galβ1-3(Fucα1-4)Glc; SAα2-3Galβ1-4GlcNAcβ1-3Galβ1-3(Fucα1-4)GlcNAcβ1-3Galβ1-4(Fucα1-3)Glc; SAα2-6Galβ1-4GlcNAcβ1-3Galβ1-3(Fucα1-4)GlcNAcβ1-3Galβ1-4(Fucα1-3)Glc; SAα2-3Galβ1-4(Fucα1-3)GlcNAcβ1-3Galβ1-3(Fucα1-4)GlcNAcβ1-3Galβ1-4(Fucα1-3) Glc; and SAα2-6Galβ1-4(Fucα1-3)GlcNAcβ1-3Galβ1-3(Fucα1-4)GlcNAcβ1-3Galβ1-4(Fucα1-3)Glc, where SA is sialic acid.

Preferably, S′ is selected from the group consisting of planar sterols. More preferably, S′ is selected from the group consisting of: campesterol, cholesterol, ergosterol, sitosterol and stigmasterol. Most preferably, S′ is cholesterol.

In a first preferment of the first aspect of the invention the water dispersible construct is of the structure:

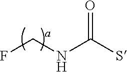

where a is the integer 2, 3 or 4.

In a second preferment of the first aspect of the invention the water dispersible construct is of the structure:

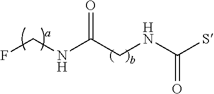

where a is the integer 2, 3 or 4 and b is the integer 3, 4 or 5.

In a third preferment of the first aspect of the invention the water dispersible construct is of the structure:

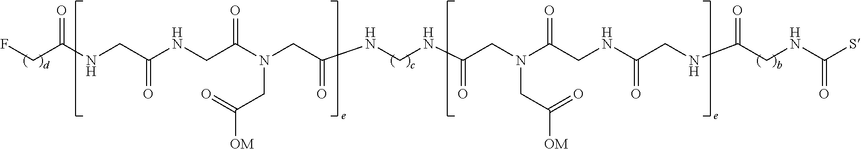

where a is the integer 2, 3 or 4, b is the integer 3, 4 or 5, c is the integer 1, 2 or 3, d is the integer 3, 4 or 5, e is the integer 1, 2 or 3 and M is a monovalent cation.

In an embodiment of the first preferment of the first aspect of the invention the water dispersible construct is of the structure:

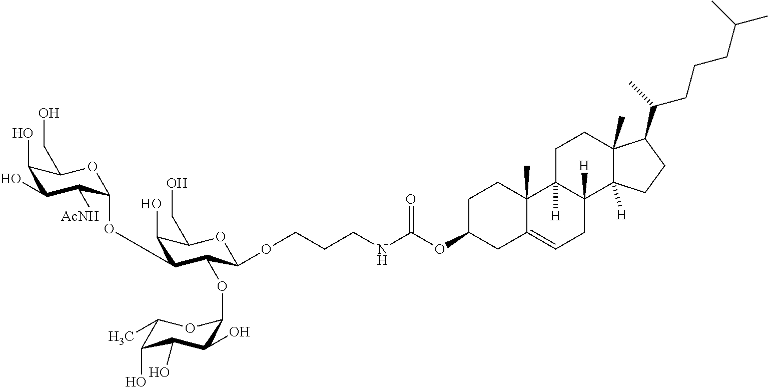

designated Aₜᵣᵢ-S₁-chol (I).

In an embodiment of the second preferment of the first aspect of the invention the water dispersible construct is of the structure:

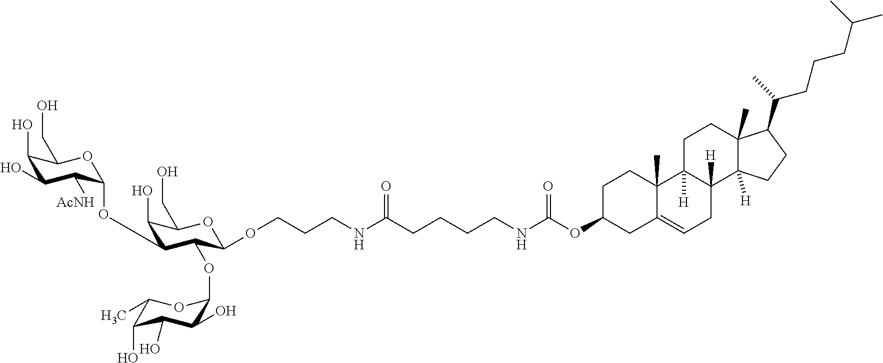

designated Aₜᵣᵢ-S₁-S₂-chol (II).

In an embodiment of the third preferment of the first aspect of the invention the water dispersible construct is of the structure:

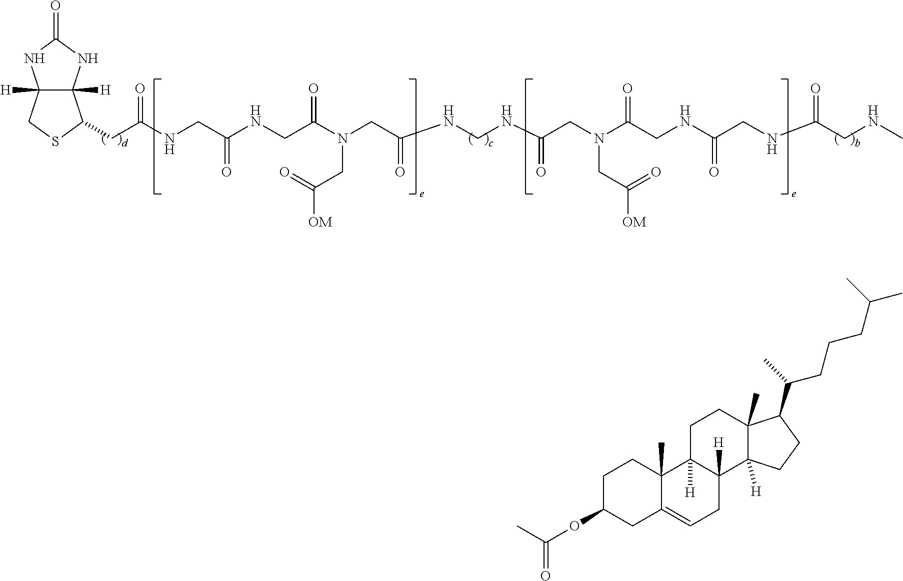

where b is 4, c is 2, d is 4, e is 2, M is sodium ion (Na⁺) and designated Biotin-S₃-S₄-S₂-chol (III).

In a fourth preferment of the first aspect of the invention, the surface is the membrane of a cell. Typically, the contacting is with a suspension of cells.

In a fifth preferment of the first aspect of the invention, the surface is the membrane of an enveloped virus. Typically, the contacting is with a suspension of enveloped viruses.

In a sixth preferment of the first aspect of the invention, the surface is the lipid bilayer of a liposome or virosome. Typically, the contacting is with a dispersion of the liposomes or virosomes.

The step of contacting the surface with an aqueous solution of a water dispersible construct may be by propelling droplets of the aqueous solution from a plurality of orifices located in a monolithic print head of an inkjet printer onto at least one discrete area on the surface.

Preferably, the volume of each of the droplets is 1 to 100 picolitres (pL). More preferably, the volume of each of the droplets is 1 to 50 pL. Most preferably, the volume of each of the droplets is 1 to 5 pL.

Preferably, the concentration of the water dispersible construct in the aqueous solution is 1 μmolar (μM) to 10 mmolar (mM). More preferably, the concentration of the construct in the aqueous solution is 10 μM to 10 mM. Most preferably, the concentration of the construct in the aqueous solution is 0.1 to 10 mM.

In a first preferment of the step of contacting the surface with an aqueous solution, the at least one discrete area is in the shape of a symbol. Preferably, the at least one discrete area is in the shape of a symbol readable by optical character recognition (OCR) apparatus. More preferably, the at least one discrete area is in the shape of a symbol comprising one or more alphanumeric characters. In a second preferment of the step of contacting the surface with an aqueous solution, the at least one discrete area is a pattern comprising a combination of indicia to which the aqueous solution is applied at different densities (amount per unit area). The first and second preferments are not mutually exclusive.

Methods including the step of contacting a surface with an aqueous solution of a water dispersible construct by propelling droplets of a aqueous solution from a plurality of orifices located in a monolithic print head of an inkjet printer onto at least one discrete area on a surface are described in the specification accompanying international application no. PCT/NZ2010/000127 (publ. no WO 2011/002310) (Bovin et al (2011)).

In a second aspect the invention provides an aqueous solution of a water dispersible construct of the structure:

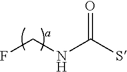

where F is a biofunctional moiety, S′ is sterol, and a is the integer 2, 3 or 4.

In an embodiment of the second aspect of the invention the water dispersible construct is of the structure:

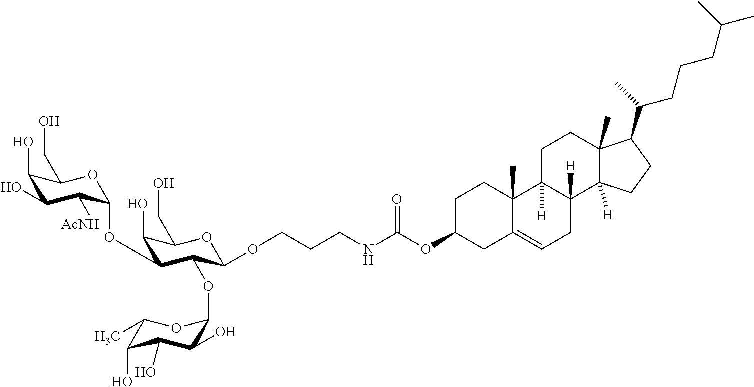

designated Aₜᵣᵢ-S₁-chol (I).

In a third aspect the invention provides an aqueous solution of a water dispersible construct of the structure:

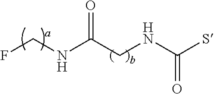

where F is a biofunctional moiety, S′ is sterol, a is the integer 2, 3 or 4 and b is the integer 3, 4 or 5.

In an embodiment of the third aspect of the invention the water dispersible construct is of the structure:

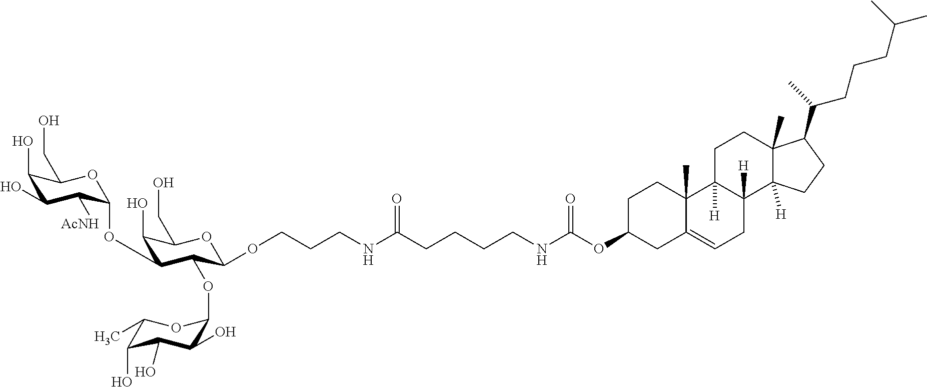

designated Aₜᵣᵢ-S₁-S₂-chol (II).

In a fourth aspect the invention provides an aqueous solution of a water dispersible construct of the structure:

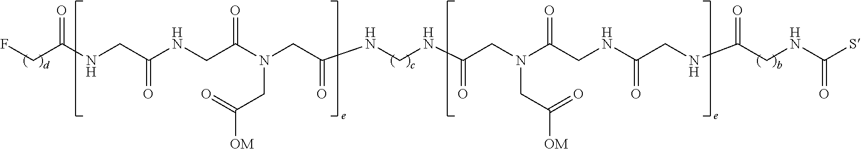

where F is a biofunctional moiety, S′ is sterol, a is the integer 2, 3 or 4, b is the integer 3, 4 or 5, c is the integer 1, 2 or 3, d is the integer 3, 4 or 5, e is the integer 1, 2 or 3 and M is a monovalent cation.

In an embodiment of the fourth aspect of the invention the water dispersible construct is of the structure:

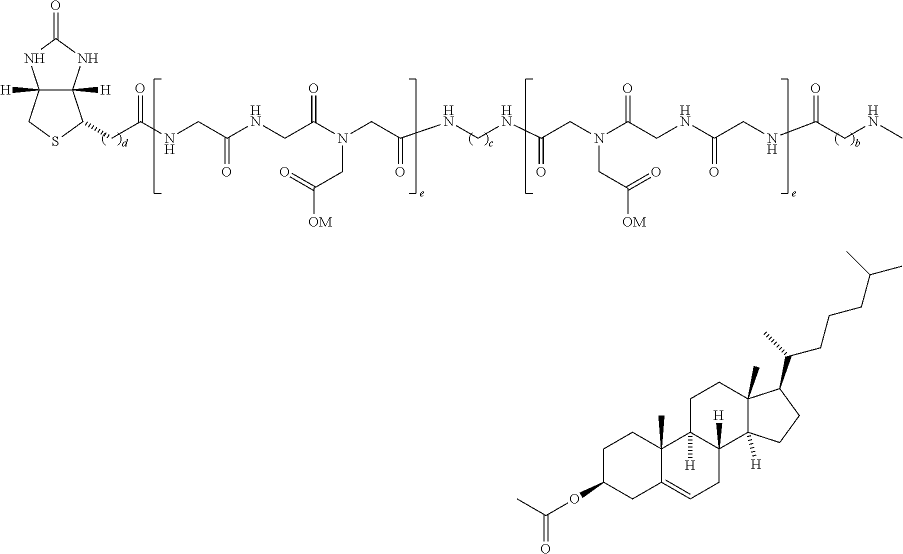

where b is 4, c is 2, d is 4, e is 2, M is sodium ion (Na⁺) and designated Biotin-S₃-S₄-S₂-chol (III).

In a fifth aspect the invention provides a cell incorporating in its membrane a water dispersible construct as defined in the second, third or fourth aspects of the invention.

In a sixth aspect the invention provides an enveloped virus incorporating in its membrane a water dispersible construct as defined in the second, third or fourth aspects of the invention.

In a seventh aspect the invention provides a liposome or virosome incorporating in its lipid bilayer a water dispersible construct as defined in the second, third or fourth aspects of the invention.

In an eighth aspect the invention provides a biofunctionalized surface incorporating a water dispersible construct as defined in the second, third or fourth aspects of the invention.

In the description, claims and representations of this specification the following acronyms, symbols, terms and phrases have the meaning provided:

“Aqueous solution” means a stable, monophasic dispersion prepared in water without the use of detergents or organic solvents at a temperature of 25° C.

“Biofunctional” means capable of participating in an interaction with one or more biomolecules in vitro or in vivo and “biofunctionalized” has a corresponding meaning. The term “biofunctional” specifically excludes covalent bond forming chemically reactive functional groups (e.g. thiols) and reporter groups (e.g. photophores).

“Biosurface engineering” means the modification of a surface by the localisation of biofunctional moieties.

“Detergent” means a substance which has the effect of altering the interfacial tension of water and other liquids or solids, but excludes the constructs of the generic structure F-S-S′ as defined herein.

“Differential expression” means the expression of the same moieties at a surface in different conformations.

“Differential localisation” means the localisation of the same or different moieties to a surface with different dissociation constants.

“Kodecyte” means a cell modified by incorporation into the cell membrane of a construct of the general structure F-S-L (where F is a functional moiety, S is a spacer selected to provide a water dispersible construct and L is a lipid).

“Kodesome” means a liposome or virosome modified by incorporation into the lipid bilayer (membrane) of a construct of the general structure F—S-L (where F is a functional moiety, S is a spacer selected to provide a water dispersible construct and L is a lipid).

“Kodevirion” means an enveloped virus particle modified by incorporation into the enveloping membrane of a construct of the general structure F-S-L (where F is a functional moiety, S is a spacer selected to provide a water dispersible construct and L is a lipid).

“Localised” means associated with a surface by non-covalent interactions and “localising” and “localisation” have a corresponding meaning.

“Monovalent cation (M)” means a counter ion having a positive charge of one (+1).

“Oligosaccharide” means a saccharide containing two or more monosaccharide units, typically two to ten monosaccharide units.

“( )^{x}”, “( )ₓ”, “[ ]ₓ” and “[ ]^{x}” mean the group contained in the parentheses is repeated a number (x) of times. By way of illustration:

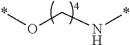

means the methylene group (—CH₂—) is repeated 4 times and the structure represented is equivalent to:

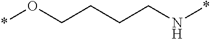 [SMILES: *NCCCCO*]

It will be understood that where the terms used to define F, S and S′ refer to molecules, the corresponding radical is comprised in the amphipathic construct (F-S-S′). For example, where the term “cholesterol” is used to define S′ it will be understood that the radical cholesteryl is comprised in the amphipathic construct F-S-S′. Unless otherwise stated S is a spacer covalently linking F to S′ via the 3β-hydroxyl position of sterol (5′).

It will also be understood that that where reference is made to a step of contacting a surface with an aqueous solution of a water dispersible construct the surface is pre-formed. The contacting is for a time and at a temperature sufficient (typically 1 to 24 hours and 4 to 37° C., respectively) to achieve the localisation. The methods of localizing a biofunctional moiety to a surface including this step are to be distinguished from methods of incorporating a construct into the lipid bilayer (membrane) of a liposome or virosome by mixing an amount of construct with other components of the bilayer before preparing the liposomes or virosomes.

The terms “first”, “second”, “third”, etc. used with reference to elements, features or integers of the subject matter defined in the Statement of Invention and Claims, or when used with reference to alternative embodiments of the invention are not intended to imply an order of preference.

The invention will now be described with reference to embodiments or examples and the figures of the accompanying drawings pages.

### BRIEF DESCRIPTION OF DRAWINGS

FIG. 1. Comparison of the structures of the constructs designated Aₜᵣᵢ-S₁-chol (I)[top], Aₜᵣᵢ-S₁-S₂-chol (II)[bottom].

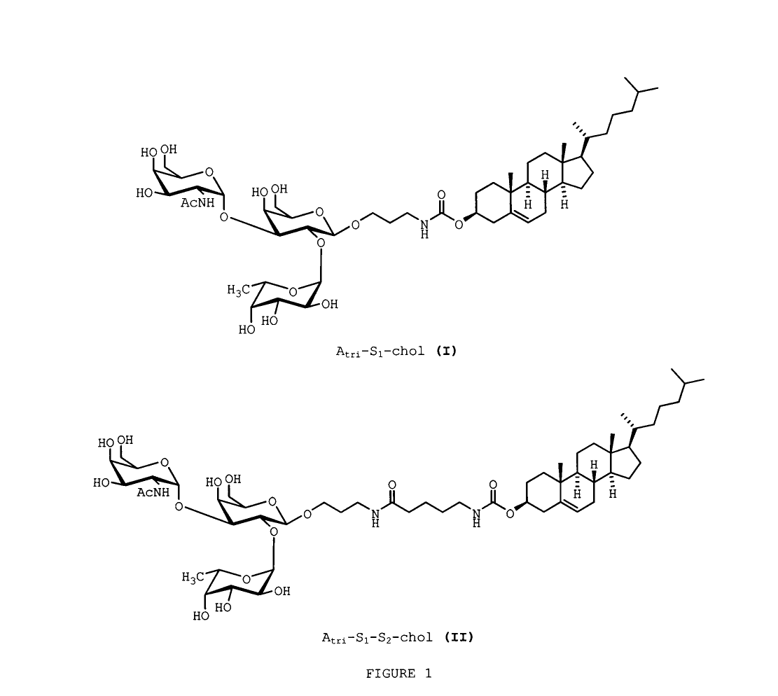

FIG. 2. Comparison of the structures of the constructs designated Biotin-S₃-S₄-S₂-chol (III)[top] and Biotin-S₃-S₄-S₅-DOPE (IV)[bottom].

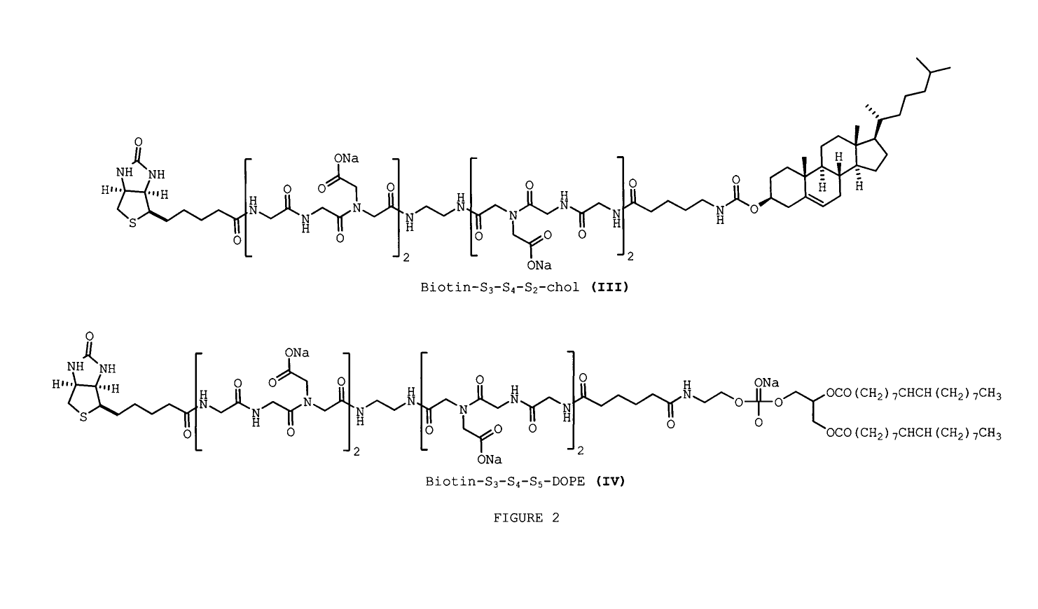

FIG. 3. ¹H NMR spectrum (3 mg/mL in CD₃OD/D₂O 2:1, 303K, 700 MHz) of the construct designated Biotin-S₃-S₄-S₂-chol (III).

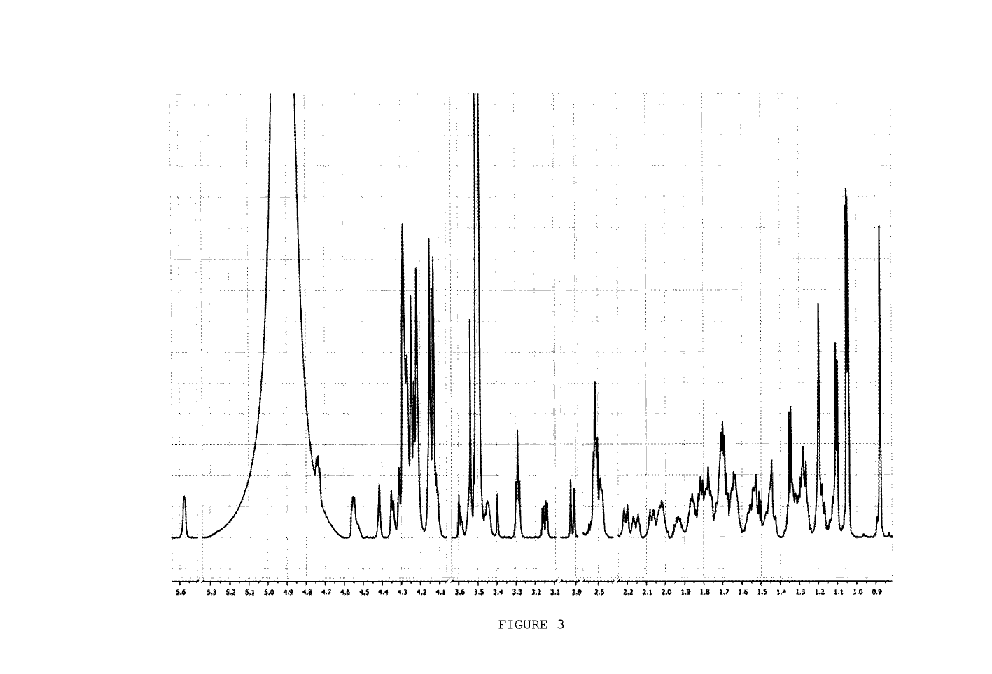

FIG. 4. Immunostaining with monoclonal antibody of an artificial surface printed with an aqueous solution of Aₜᵣᵢ-S₁-S₂-chol (II) (“FSS-A”) and Aₜᵣᵢ-sp-Ad-DOPE (“FSL-A”; Bovin et al (2005)). The surface is identified by the words appearing following immunostaining: silica gel with plastisicer (A), silica gel without plastisicer (B), nitrocellulose (C), Impress Gloss (Spicers)(D), G-Print Matt (Spicers)(E), Sapphire Cast Coated (Spicers)(F) and uncoated printer paper (G).

FIG. 5. Immunostaining with monoclonal antibody of an artificial surface (9 Lives (Spicers) or nitrocellulose) printed with an aqueous solution of Biotin-S₃-S₄-S₂-chol (III)(“FSS-Biotin”) and Biotin-S₃-S₄-S₅-DOPE (IV)(“FSL-Biotin”) at a range of concentrations (pM). The surface and concentration used is identified by the words appearing following immunostaining: 9 Lives and 5 μM FSL-Biotin (A), nitrocellulose and 5 μM FSL-Biotin (B), 9 Lives and 25 μM FSL-Biotin (C), nitrocellulose and 25 μM FSL-Biotin (D), 9 Lives and 100 μM FSL-Biotin (E), nitrocellulose and 100 μM FSL-Biotin (F), 9 Lives and 5 μM FSS-Biotin (G), nitrocellulose and 5 μM FSS-Biotin (H), 9 Lives and 25 μM FSS-Biotin (I), nitrocellulose and 25 μM FSS-Biotin (J), 9 Lives and 100 μM FSS-Biotin (K), nitrocellulose and 100 μM FSS-Biotin (L).

FIG. 6. Photomicrographs of kodecytes prepared using aqueous solutions of the constructs designated Biotin-S₃-S₄-S₂-chol (III) (C, D, G, and H) and Biotin-S₃-S₄-S₅-DOPE (IV) (A, B, E, and F) at concentrations of 5 μM (A-D) and 10 μM (E-H). Upper field (A, C, E and G)—fluorescence microscopy following conjugation with avidin-ALEXAFLUOR™. Lower field (B, D, F and H)—morphology of cells under light microscopy.

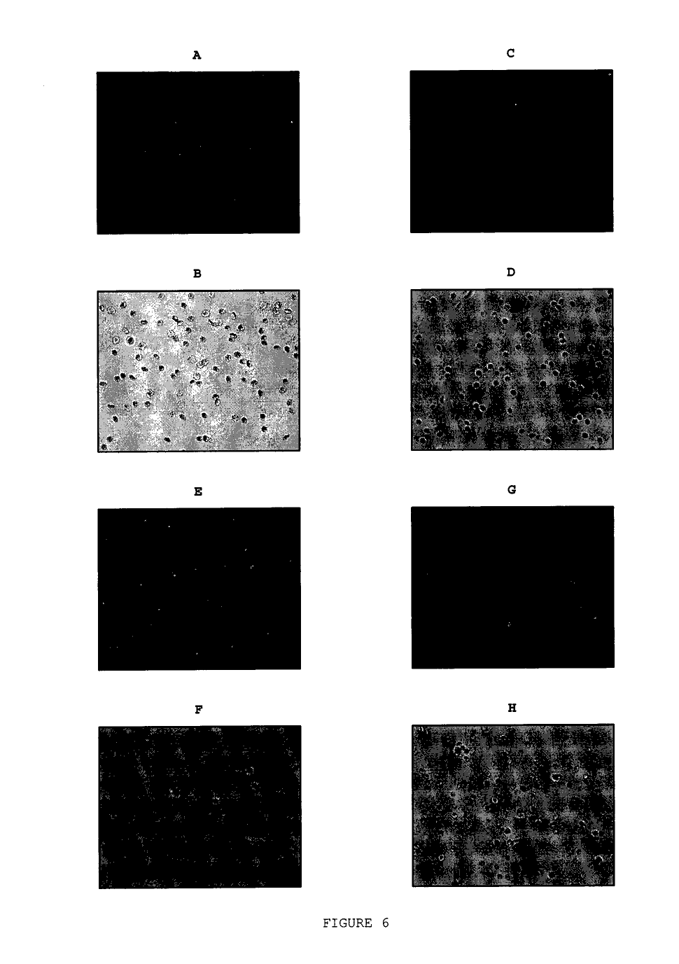

FIG. 7. Photomicrographs of kodecytes prepared using aqueous solutions of the constructs designated Biotin-S₃-S₄-S₂-chol (III) (C, D, G, and H) and Biotin-S₃-S₄-S₅-DOPE (IV) (A, B, E, and F) at concentrations of 25 μM (A-D) and 50 μM (E-H). Upper field (A, C, E and G)—fluorescence microscopy following conjugation with avidin-ALEXAFLUORm. Lower field (B, D, F and H)— morphology of cells under light microscopy.

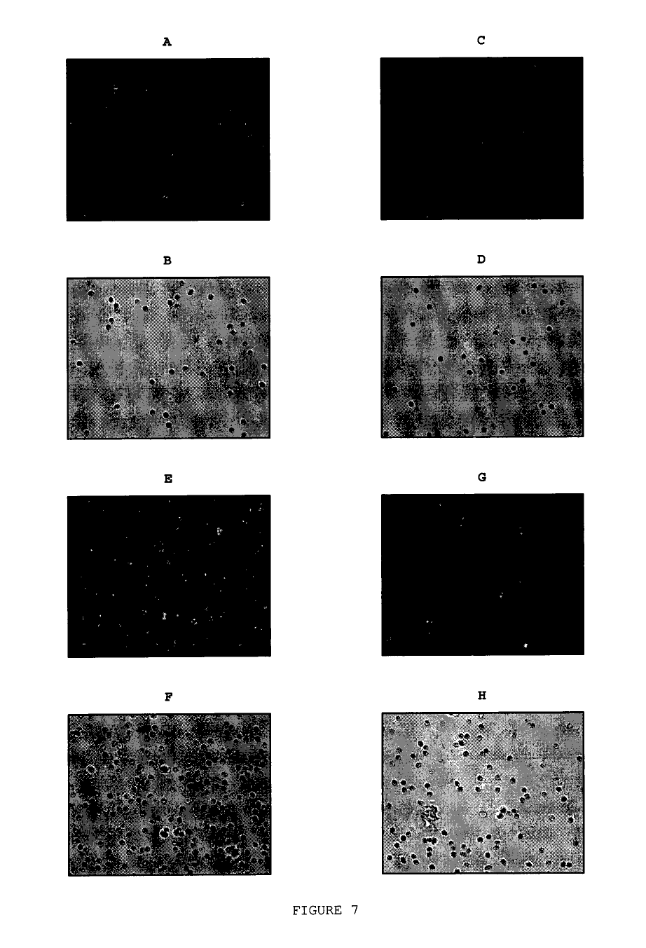

FIG. 8. Photomicrographs of kodecytes prepared using aqueous solutions of the constructs designated Biotin-S₃-S₄-S₂-chol (III) (C, D, G, and H) and Biotin-S₃-S₄-S₅-DOPE (IV) (A, B, E, and F) at concentrations of 75 μM (A-D) and 100 μM (E-H). Upper field (A, C, E and G)—fluorescence microscopy following conjugation with avidin-ALEXAFLUOR™. Lower field (B, D, F and H)—morphology of cells under light microscopy.

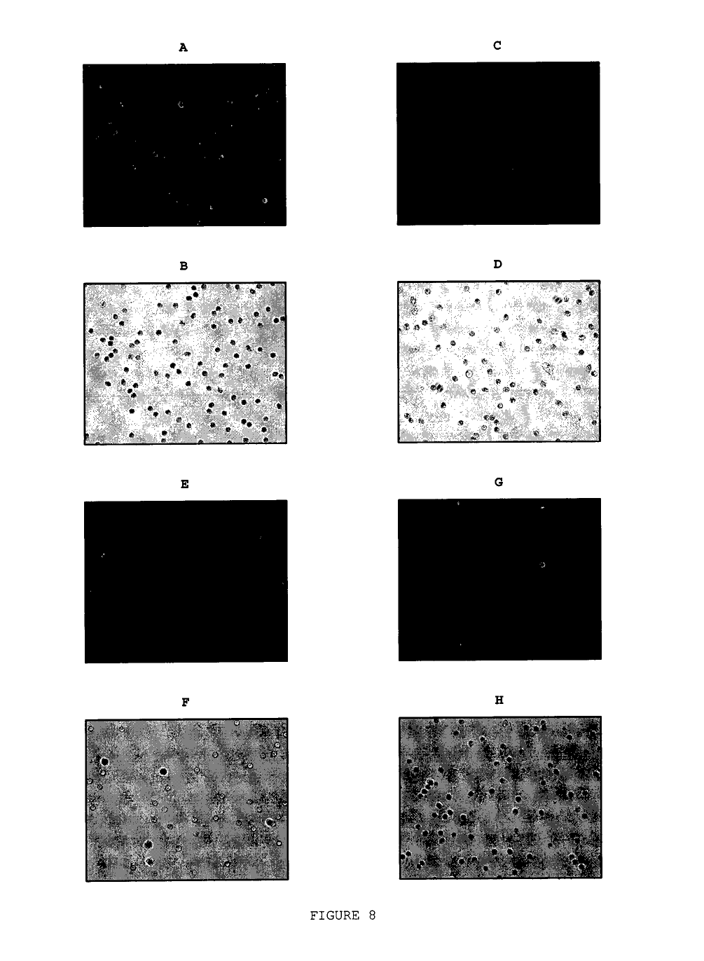

FIG. 9. Photomicrographs of kodecytes prepared using aqueous solutions of the constructs designated Biotin-S₃-S₄-S₂-chol (III) (C and D) and Biotin-S₃-S₄-S₅-DOPE (IV) (A and B) at a concentration of 500 μM. Upper field (A and C)—fluorescence microscopy following conjugation with avidin-ALEXAFLUOR™. Lower field (B and D)—morphology of cells under light microscopy.

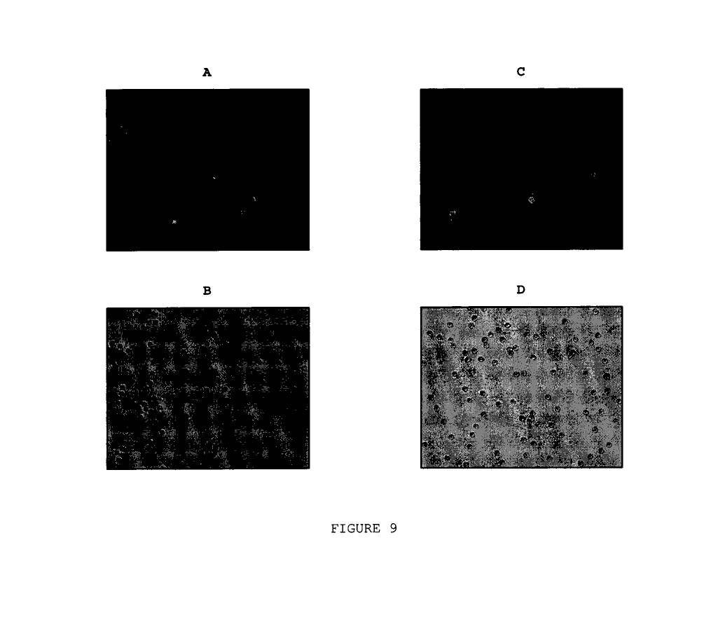

FIG. 10. Photomicrographs of kodecytes prepared using aqueous solutions of the constructs designated Biotin-S₃-S₄-S₂-chol (III) (C, D, G, H, K, L, O, P, S and T) and Biotin-S₃-S₄-S₅-DOPE (IV) (A, B, E, F, I, J, M, N, Q and R) at concentration of 0 μM (A-D), 5 μM (E-H), 25 μM (1-L), 50 μM (M-P) and 100 μM (Q-T) and mixed with avidinylated magnetic beads. Left field (A, C, E, G, I, H, M, O, Q and S) at 400× magnification. Right field (B, D, F, H, J, L, N, P, R and T) at 800× magnification.

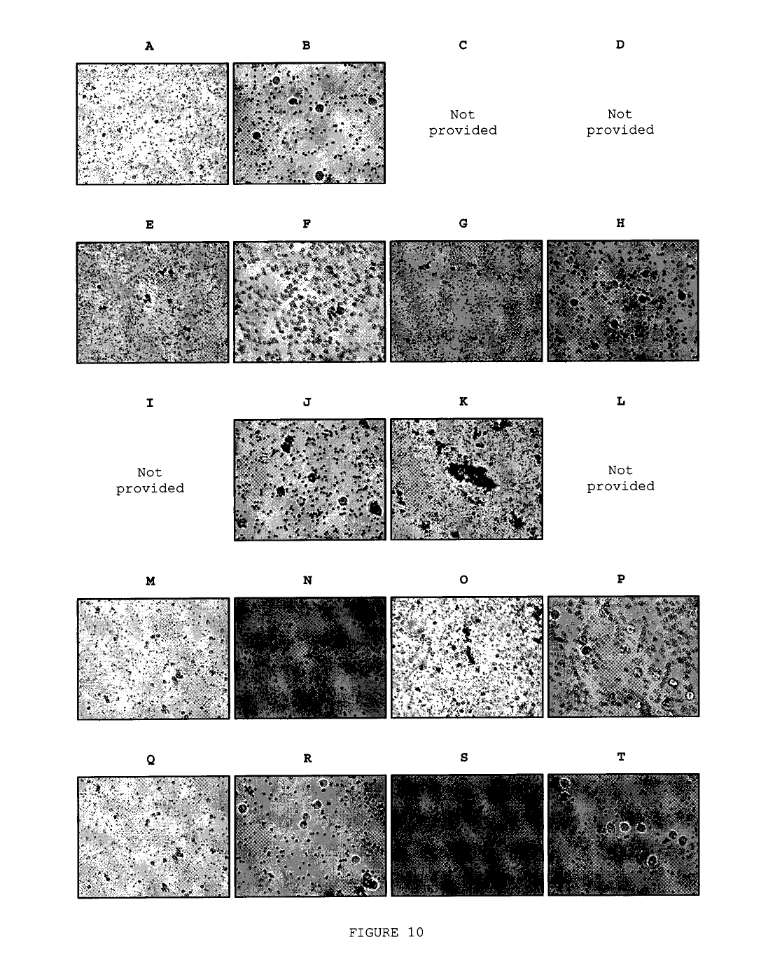

FIG. 11. The structures of the constructs designated Aₜᵣᵢ-S₁-S₅-DOPE (V) [top] and Aₜᵣᵢ-S₁-S₅-S₄-S₅-DOPE (VI)[bottom].

### DETAILED DESCRIPTION

The invention uses constructs of the generic structure F-S-S′ where S′ is a sterol. These water dispersible constructs are readily dispersible in water and may be used in conjunction with their diacyl or dialkyl counterparts (as described in the publications of Bovin et al (2005), Korchagina et al (2008), Bovin et al (2009) and Weinberg et al (2009)) to provide biofunctionalized surfaces and surface modified biological structures with novel characteristics. Where the surface is a lipid bilayer (membrane), such as the surface of a liposome or virosome, the sterol (S′) of the construct has the potential to stabilise and improve the structural integrity of the membrane. The amphipathic constructs may therefore be used advantageously in the preparation of such vehicles and biomimetic films such as those described in the publication of Spedden (2008).

Water soluble constructs of the generic structure F-S-L where L is a phospholipid are described in the publications of Bovin et al (2005), Korchagina et al (2008), Bovin et al (2009) and Weinberg et al (2009). The primary amino (NH₂—) function of phosphatidylethanolamine provides a convenient means by which functional moieties (F) can be conjugated to the lipid (L) via an appropriate spacer (S). Methods of conjugating specific functional moieties to sterol are described in the publications of Barragan et al (2001), Boonyarattanakalin et al (2006), Booth et al (2001), Carrion et al (2001), DUffels et al (2000), Orr et al (1979), Pacuszka et al (1991), Spencer et al (2004), Vidal et al (2003) and others.

The publication of Barragan et al (2001) describes the synthesis of mannose-6-phosphonate-cholesterylamine conjugates via 3-aminocholesterol. The publication of Booth et al (2001) discloses the synthesis of biotinylated cholesteryl derivatives. The publication of Carrion et al (2001) discloses the preparation of poly(ethylene glycol)-coated liposomes using pegylated cholesterol derivatives linked either directly or through a spacer arm of diaminebutane via a carbamate bond. The publication of Duffels et al (2000) discloses the synthesis of high mannonse type cholesterol-based neoglycolipids. The publication of Orr et al (1979) describes the preparation of synthetic mannose-containing glycolipids utilising the cholesterol nucleus as a lipid anchor and either a 6-aminohexyl- or a 6-(6-aminohexanamido) hexyl-1-thio-α-D-mannopyranoside as a carbohydrate ligand. The publication of Pacuszka et al (1991) discloses the synthesis of several lipid analogues of ganglioside G_{M1} including an analog prepared by attaching the oligosaccharide moiety of the ganglioside to cholesteryl hemisuccinate.

The water soluble constructs used in the present invention are prepared via cholesterol chloroformate. The poor dispersibility of sterol chloroformate (relative to phosphatidyl ethanolamine, e.g. DOPE) in solvents compatible with the dissolution of very hydrophilic derivatives of functional moieties such as biotin-S₃-S₄ and Aₜᵣᵢ-S₁ results in negligible yield of water soluble construct. This incompatibility, arising from the hydrophilicity of the derivative of the functional moiety and hydrophobicity of the sterol derivative and the resulting heterogeneous reaction mixture, presents a technical obstacle to the facile synthesis of the water soluble constructs. The use of a steroylcarbonylaminoalkanoic acid Nos-succinimide esters, such as that prepared according to Scheme I, in the preparation of water soluble constructs, such as that prepared according to Scheme IV, has been found to be advantageous in promoting yields where the derivative of the functional moiety is particularly hydrophilic.

In the publication of Pacuszka et al (1991) although the oligosaccharide moiety provided the recognition site for the binding of cholera toxin, the nature of the lipid moiety played an important role in the action of the toxin. More recently, the publication of Lingwood et al (2011) has stated that membrane cholesterol induces a tilt in glycolipid receptor headgroups, resulting in loss of access for ligand binding. The collective behaviour of lipids is documented as a dimension of surface recognition and communication. It is suggested that lipid “allostery” can be a means to regulate membrane recognition processes. Membrane cholesterol is identified as a key molecule in the regulation of glycolipid confirmation and receptor function. The publication of Spencer et al (2004) suggested an unexpected tolerance of biological membranes regarding the incorporation of sterols of differing chemical structure based on studies using a number of benzophenone-containing cholesterol analogues notably, the analogues in which the 3β-hydroxyl group had been replaced with a large amidobenzophenone group were capable of replacing up to 50% of cellular free cholesterol.

In the publication of Duffels et al (2000) the neoglycolipids are proposed for use as additives to cationic liposome formulations in the active targeting of liposomes to macrophages. The publication of Zurbriggen et al (2006) describes a method for the effective lyophilization and reconstitution of fusion-active vehicles, namely virosomes. The virosomes prepared by the method are presented as being particularly useful to deliver biologically active substances, including antigens, drugs and other pharmaceutically active substances including DNA, RNA or siRNA into cells. The method utilises cationic lipids, specifically cationic cholesteryl derivatives. The virosomal membrane compositions comprise preferably between 1.9 and 37 mol % 3β[N—(N′,N′-dimethylammonioethane)-carbamoyl]cholesterol chloride (DC-Chol) or 3β[N—(N′,N′N′-trimethylammonioethane)-carbamoyl]cholesterol chloride (TC-Chol), the residual lipid content of the membrane consisting preferably of phospholipids.

It is recognised that the method of the present invention may be employed in conjunction with methods of preparing and/or reconstituting fusion-active vehicles, such as virosomes. In the methods described by Zurbriggen et al (2006) the functional viral envelope glycoproteins hemagglutinin (HA) and neuraminidase (NA) intercalated in the phospholipid by-layer are proposed as imparting structural stability and homogeneity to the virosomal formulations. In the method of the present invention the water dispersible constructs per se are anticipated to import such structural stability. The use of potentially destabilising organic solvents or detergents, such as octaethyleneglycol-mono-(n-dodecyl)ether (OEG), is inconsistent with realising this advantage.

It is also anticipated that the constructs used in the method of the present invention will preferentially partition into lipid rafts when incorporated into cell membranes. This preferential partitioning may be utilised to promote endocytosis or viral infection by the concentration of appropriate biofunctional moieties (ligands).

### EXPERIMENTAL

### Preparation of Aₜᵣᵢ-S₁-chol (I)

To a solution of cholesteryl chloroformate (16.8 mg, 0.0375 mmol) in dry dichloromethane (1 ml) was added Aₜᵣᵢ-S₁-NH₂ (20 mg, 0,034 mmol) in DMF (0.5 ml) and triethylamine (5 μL). The mixture was incubated for 1 hour at room temperature followed by column chromatography (Sephadex LH-20, 1:1 (v/v) chloroform-methanol) of the mixture to yield I (80%).

### Preparation of SuO-S₂-chol [Scheme I]

To a solution of cholesteroylcarbonylaminopentanoic acid (S₂-S′) (7.9 mg, 14.9 mkM) in DMF (0.3 mL), DSC (3.8 mg, 14.8 μM) and triethylamine Et₃N (2 μL, 14.4 μM) were added. The solution was stirred for 2 hours to provide a solution of cholesteroyl carbonylaminopentanoic acid Nos-succinimide ester (Nos-S₂-S′).

### Preparation of Aₜᵣᵢ-S₁-S₂-chol (II) [Scheme II]

To a solution of Nos-S₂-S′ (25 mg, 0.04 mmol) in dry N,N-dimethylformamide (1 ml) was added Aₜᵣᵢ-S₁-NH₂ (21.1 mg, 0.036 mmol) in DMF (1 ml) and triethylamine (20 μl) was added. The mixture was kept for 1 hour at room temperature followed by column chromatography (Sephadex LH-20, 1:1 (v/v) chloroform-methanol) of the mixture to yield II (78%).

### Biotin-S₃-S₄-S₂-chol (III) [Scheme IV]

To a solution of cholesteroylcarbonylaminopentanoic acid Nos-succinimide ester (Nos-S₂-S′) was added a solution of biot-S₃-S₄-NH₂ (9.1 mg, 7.17 μM) in isopropanol/H₂O (1:1 (v/v), 1 mL). The heterogeneous mixture was stirred for 15 hours before being evaporated and the residue separated on a silica gel column (15 mL) eluted with CHCl₃/MeOH (5:1 (v/v)). The target Biotin-S₃-S₄-S₂-chol (III) was eluted with CHCl₃/MeOH/H₂O (6:3:1 (v/v/v)). The yield of biotin-S₃-S₄-S₂-chol (III) was 2 mg (15.5% calculated on the basis of biot-S₃-S₄-NH₂).

¹H NMR (700 MHz, [D₂]H₂O/[D₄]CH₃OH 1:2, 30° C.): 5.575 (d, J=3.6 Hz 1H; ═CH of cholesterol), 4.737 (dd, J=7.8 Hz, J=5 Hz, 1H; NHCH of biotin), 4.553 (dd, J=7.8 Hz, J=4.3 Hz, 1H; NHCH of biotin), 4.534 (m, 1H; OCH of cholesterol), 4.470-4.078 (total 32H; 4 CH₂COO, 12 NCH₂CO), 3.540 (m, 4H; NHCH₂CH₂NH), 3.446 (m, 1H; NHCHCH of biotin), 3.290 (t, J=6.7 Hz, 2H; CH₂N of aminovaleric res.), 3.149 (dd, J=13 Hz, J=5 Hz, 1H; NHCHCH of biotin), 2.916 (dd, J=13 Hz, J<2 Hz, 1H; NHCHCH of biotin), 2.515 (m, 6H; 2 CH₂CO and ═CH—CH₂ of cholesterol), 2.208-1.105 (total 39H; 29H of cholesterol, 4H of aminovaleric res. and 6H of biotin), 1.105 (d, J=6.6 Hz, 3H; CH₃CH of cholesterol), 1.050 and 1.046 (2 d, J=6.6 Hz, 2×3H; 2 CH₃CH of cholesterol), 0.878 (s, 3H; CH₃ of cholesterol) ppm.

The structures of Aₜᵣᵢ-S₁-chol (I) and Aₜᵣᵢ-S₁-S₂-chol (II) are to be contrasted with the structure of the construct designated Aₜᵣᵢ-S₁-S₅-DOPE as described in the publication of Bovin et al (2005). The differential localisation of these constructs was demonstrated by printing on artificial surfaces.

### Ink Jet Printing of Constructs

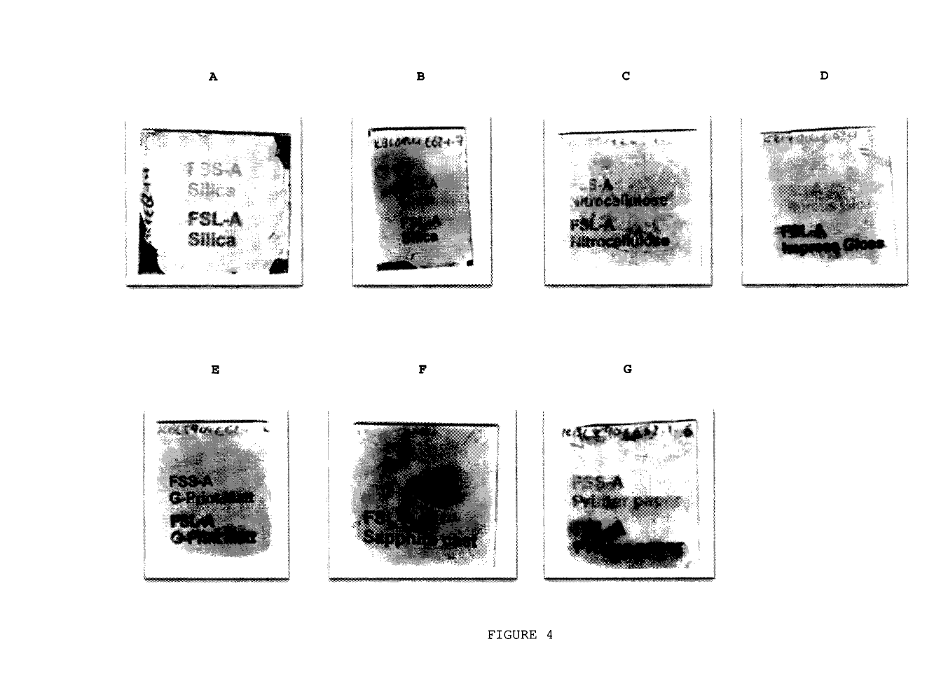

An ink jet printer (EPSON STYLUS™ T21) with refillable cartridges modified to hold a smaller volume was employed. The constructs were prepared as solutions at a concentration of circa 6 mM. Each one of the solutions was used to fill separate modified cartridges permitting both solutions to be printed at the same time on the same sample of paper. To facilitate identification the identification of the solution and artificial surface were printed. Following printing of the two solutions each sample of artificial surface was blocked with a 2% (w/v) solution of BSA and immunostained with monoclonal anti-A and then anti-mouse IgG conjugated to alkaline phosphatase and the chromogenic substrate NBT-BCIP. The immunostained samples of printed paper are presented in FIG. 4. The trial indicated that a reduced chromogenic response was obtained when the biofunctional moiety (F) Aₜᵣᵢ was localised to the artificial substrate using a construct (F-S-L) where L of the construct was a sterol (S′). The intensity of the chromogenic response also appeared to be influenced by the nature of the artificial substrate employed.

A second trial was performed employing the same methodology, but using constructs (F-S-L and F-S-S′) where the biofunctional moiety (F) of the constructs was biotin. In addition, nitrocellulose was included as an artificial substrate. As in the first trial the identification of the construct in the solution and the artificial surface were printed. On this occasion visualisation of the printed construct was achieved by conjugation with streptavidin-alkaline phosphatase conjugate (Sigma). Solutions of the construct designated Biotin-S₃-S₄-S₂-chol (III) (“FSS-Biotin”) and the construct designated Biotin-S₃-S₄-S₅-DOPE (IV) (“FSL-Biotin”) were prepared at concentrations of 5 mM, 25 mM and 100 mM in PBS containing 0.01% v/v TWEENI™ 20 and 1% magenta ink. Following printing the printed artificial surfaces were blocked with 2% (w/v) BSA in PBS for 60 minutes at room temperatures. The printed artificial surfaces was then washed by flooding the surface six times with PBS for 20 seconds each washing step. The surface of the washed artificial substrate was then flooded with a solution of 2 μg/mL of the streptavidin-alkaline phosphatase conjugate and incubated at room temperature for 30 minutes before washing with the substrate buffer (pH 9.5) containing 100 mM Tris, 100 mM sodium chloride and 50 mM magnesium chloride. Following washing with the substrate buffer the surface of the artificial substrate was flooded with a 50-fold dilution in PBS of 18.75 mg/mL nitro blue tetrazolium chloride and 9.4 mg/mL 5-bromo-4-chloro-3-indolyl phosphate, toluidine salt in 67% dimethyl sulfoxide (DMSO) (Roche).

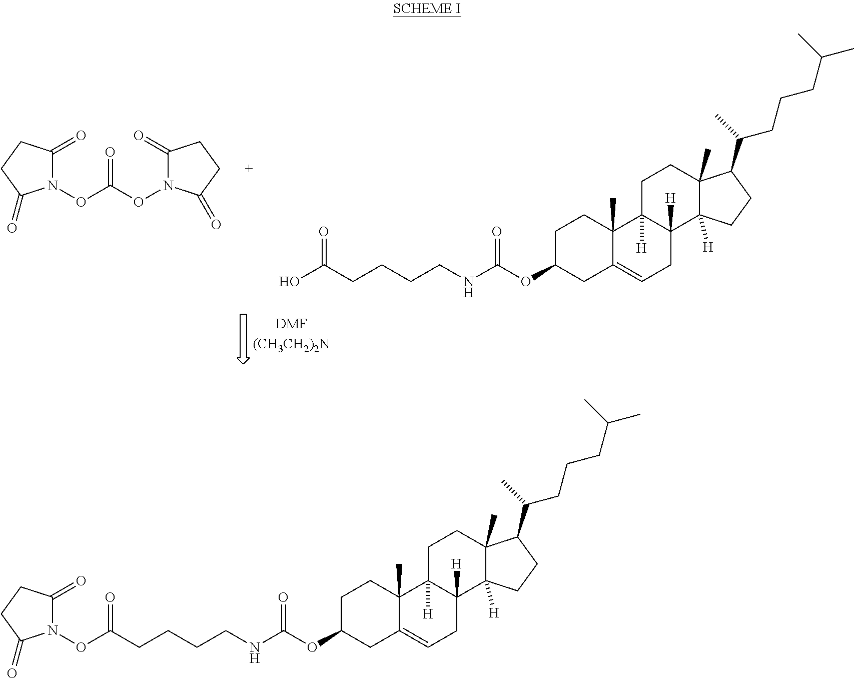

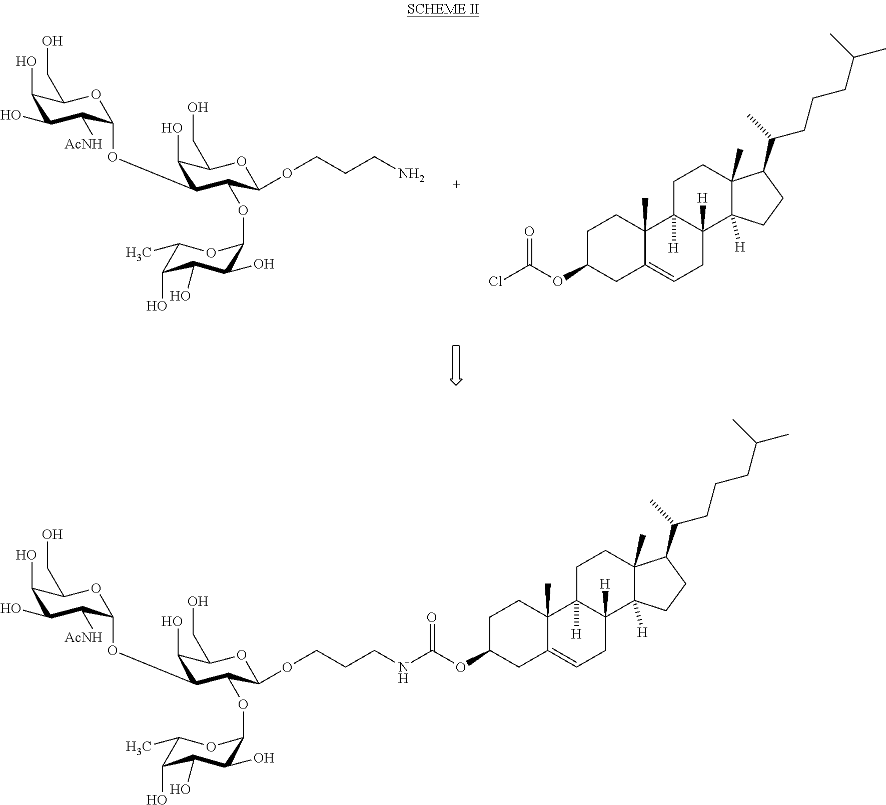

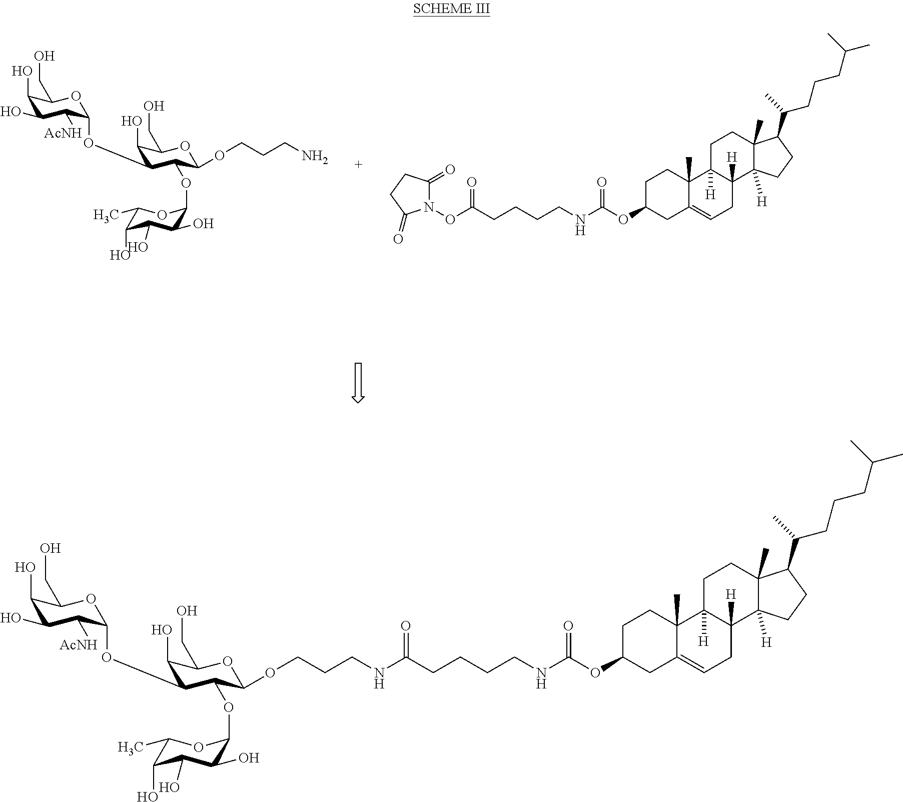

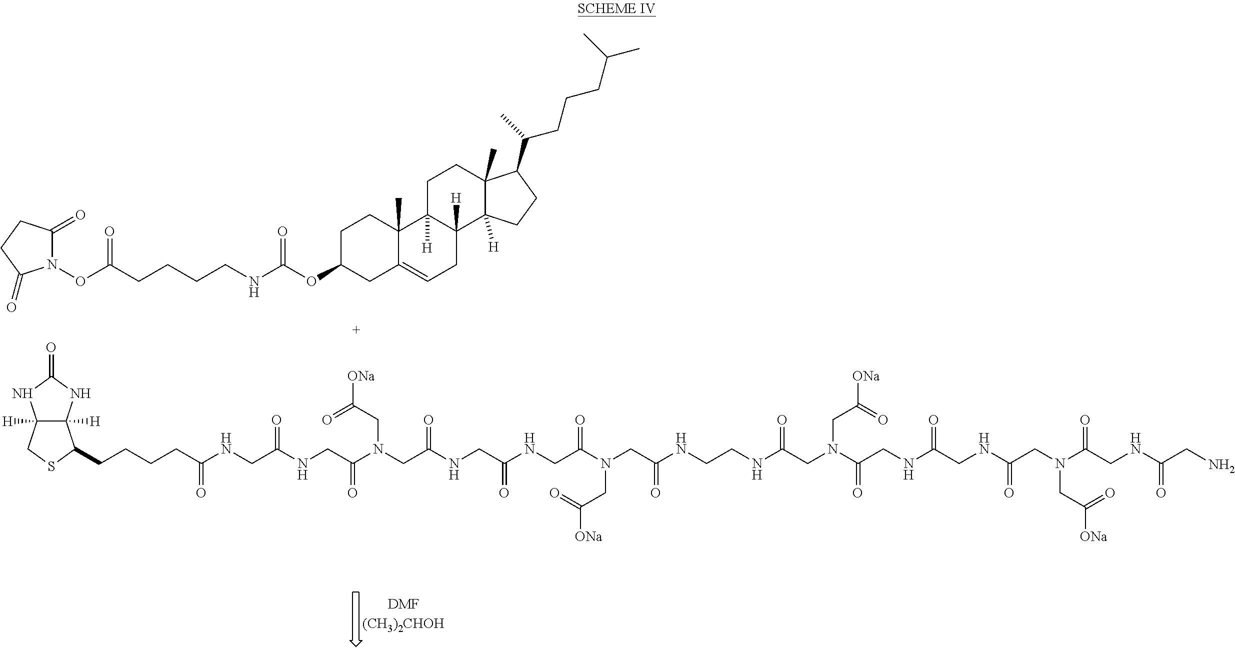

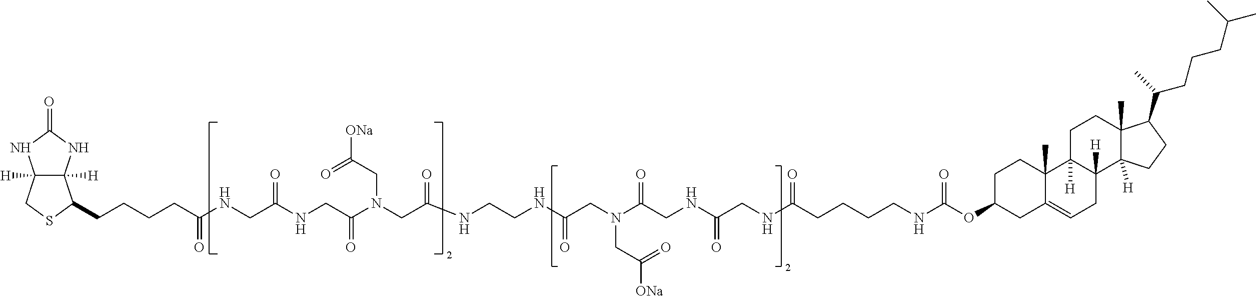

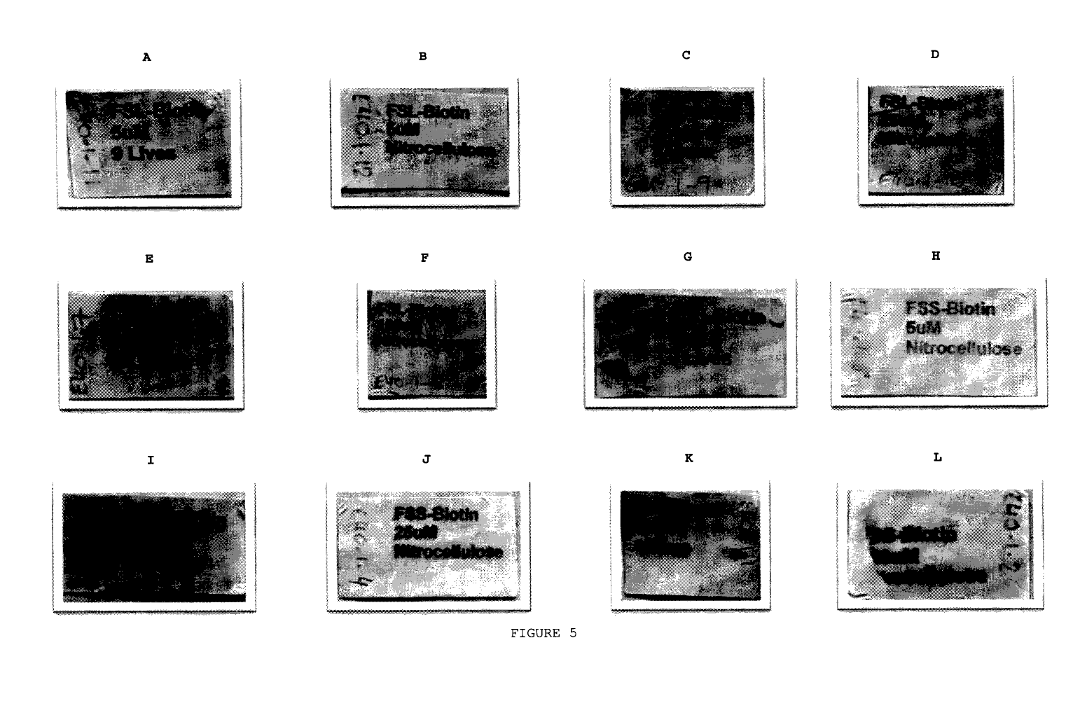

The artificial substrate was incubated at room temperature for 5 minutes before washing the surface with deionised water to stop the chromogenic reaction. The results are presented in FIG. 5.

### Modification of Cell Surfaces—Biotin as F

The use of aqueous solutions of the construct designated Biotin-S₃-S₄-S₂-chol (III) and the construct designated Biotin-S₃-S₄-S₅-DOPE (IV) in the localisation of biotin (F) to cell surfaces was also compared. Solutions of the two constructs were prepared at substantially equimolar concentrations. The use of the solutions was assessed across a concentration range from 0 to 500 μM (0.5 mM). A 0.5 mM solution of the construct designated Biotin-S₃-S₄-S₂-chol (III) was prepared by dissolving 0.4 mg of the construct in 514 μL of PBS. The construct readily dissolved to provide a clear solution. A 0.5 mM stock solution of the construct designated Biotin-S₃-S₄-S₅-DOPE (IV) was prepared by dissolving 2 mg of the construct in 1942 μL of phosphate buffered saline (PBS). Each 0.5 mM solution was warmed to 37° C. for 10 minutes, vortexed and sonicated for 30 seconds to ensure complete dissolution of the construct in the aqueous medium. A dilution series was prepared for each construct to provide a total volume of 100 μL at each of the following concentrations: 5, 10, 25, 50, 75, 100 and 500 LM. Equal volumes (40 μL) of packed, washed red blood cells (RBCs) and solutions of constructs at different concentrations were mixed in a glass kimble tube. The tubes were incubated at 37° C. for 20 minutes in a water bath with periodic (3 x) agitation. The incubated cells were washed 3× with PBS before being resuspended in a cell preservative (CELPRESOL™) for storage at 4° C. overnight.

Two equal volumes were taken from each tube and centrifuged (300×g for 2 minutes) to provide two equivalent packed cells volumes for further evaluation. One of each pair of equivalent packed cell volumes was evaluated for localisation of the biofunctional moiety biotin to the surface of the RBCs by conjugation with avidin ALEXAFLUOR™. One of each pair of the equivalent packed cell volumes was evaluated for localisation of the biofunctional moiety biotin to the surface of the RBCs by conjugation to avidinylated beads (DYNABEADS™ M-280).

Conjugation with avidin ALEXAFLUOR™ was evaluated as described in the publication of Oliver et al (2011). An aliquot containing 0.02 mg avidin ALEXAFLUOR™ was mixed with a volume (190 μL) of PBS containing BSA to provide a 0.1 mg/mL solution. Equal volumes 20 μL of packed RBCs and the solution of avidin ALEXAFLUOR™ were mixed and incubated at 37° C. for 30 minutes. The cells were then washed 3× with PBS before placing a drop (10 μL) on a glass microscope slide for fluorescent microscopy (WIB filter at 400× magnification). Cells were graded for fluorescence intensity over two micrograph fields of view. Grades determined via the eye piece were correlated with the corresponding photomicrographs (1.9 sec exposure). Grades were assigned according to the following scale: 1 (low), 2 (moderate), 3 (bright) and 4 (very bright). The morphology of the cells was also recorded (“normal” or “poiked”).

Conjugation with avidinylated beads (DYNABEADS™ M-280) was performed. An aliquot of a volume (100 μL) of beads (1 μm diameter, 12×10⁹ beads per mL) was washed 3× in sterile PBS for one minute at 1000×g. The concentration of the beads was adjusted to 6×10⁹ beads/mL with 100 μL PBS and a volume (5 μL) mixed with an equal volume of a 1% dilution of packed RBCs (circa 7.5×10⁷ cells/mL) directly on a microscope slide. The ratio of beads to cells was approximately 600. This ratio of beads to cells was easily mixed by drawing the volume in and out of the pipette tip before overlaying a coverslip. The attachment of beads to cells was scored at 400× and 800× magnification on a compound microscope. An estimate of the number of beads and the presence of cell clusters was recorded for each group and compared with a negative control.

The fluorescent intensity determined for cells prepared using either the construct designated Biotin-S₃-S₄-S₅-DOPE (IV) or the construct designated Biotin-S₃-S₄-S₂-chol (III) was generally similar. The use of the construct designated Biotin-S₃-S₄-S₂-chol (III) at lower concentration ranges (5 to 75 μM) provided moderately lower fluorescent signal intensity relative to cells prepared using the construct designated Biotin-S₃-S₄-S₅-DOPE (IV) over the same concentration range. However, cells prepared using the construct designated Biotin-S₃-S₄-S₂-chol (III) appeared to show a more homogenous modification over the entire cell surface and lesser inter-cell variation. This may indicated that the modification of cells with the construct designated Biotin-S₃-S₄-S₂-chol (III) is less influenced by intra and inter-cell variation in the composition of the cell membrane. Both of the constructs were observed to show poor staining at the higher concentrations of 100 and 500 μM with both intra and inter-cell variation. The use of the constructs at concentrations of 10 and 25 μM appeared to provide the most consistent and uniform modification with a fluorescent signal intensity of grade 2. The use of the construct designated Biotin-S₃-S₄-S₅-DOPE (IV) and the construct designated [FSS-Biotin]produced comparable results when evaluated by attachment of avidinylated beads (DYNABEADS™ M-280).

The attachment of beads was observed to be optimal when 25 μM concentrations of the two constructs were used. At this concentration 4 to 8 beads were observed to be attached to each RBC along with clustering of RBCs in groups of 8 or more cells. The number of beads attached to each RBC and the clustering of RBCs into groups was observed to be slightly greater where these cells were prepared using the construct designated Biotin-S₃-S₄-S₂-chol (III). Similarly, optimum results were obtained when concentrations of the constructs in the range 10 to 25 μM were used. This suggests that the uniformity of modification of the cells surface is a more significant determinant when evaluating bead attachment than level of modification (cf. fluorescent signal intensity). A reduced level of attachment and the absence of clustering was observed when the constructs were used at concentrations of 100 and 500 μM. It is suggested that at these higher concentrations the association of the constructs with the surface of the cells is in the form of micelles rather than by insertion into the outer cell membrane. It is concluded that both constructs may be used optimally at concentrations in the range 10 to 50 μM.

### Modification of Cell Surfaces—A_{Tri} as F

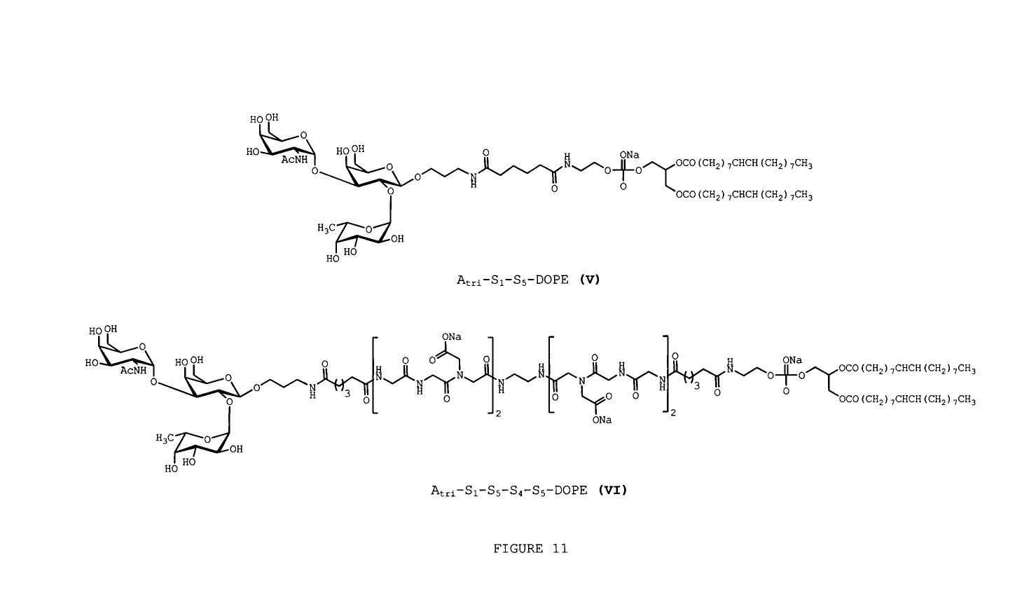

The use of aqueous solutions of the construct designated Aₜᵣᵢ-S₁-chol (I), the construct designated Aₜᵣᵢ-S₁-S₅-DOPE (V) and the construct designated Aₜᵣᵢ-S₁-S₅-S₄-S₅-DOPE (VI) in the localisation of Aₜᵣᵢ (F) to cell surfaces was also compared. (The constructs designated Aₜᵣᵢ-S₁-S₅-DOPE (V) and Aₜᵣᵢ-S₃-S₄-S₅-DOPE (VI) (FIG. 11) were prepared according to Bovin et al (2005) and Bovin et al (2009)). Volumes (40 μL) of packed, washed O group red blood cells (RBCs) were resuspended in equal volumes of solutions of each of the three constructs at a concentration of 200 μg/mL in PBS. The resuspended RBCs were incubated at room temperature for 4 hours prior to storage at 4° C. overnight. Serology was performed as described by Bovin et al (2005) using a range of monoclonal preparations and sera. The monoclonal preparations and sera used and serology scores obtained are listed in Table 1. Notably only certain monoclonal preparations and sera where cross-reactive with the kodecytes prepared using the construct designated Aₜᵣᵢ-S₁-chol (I). These results are consistent with insertion of the construct, but presentation of the glycotope (Aₜᵣᵢ) at the cell surface in a confirmation not capable of cross-reacting with all anti-A or anti-A/B monoclonal preparations and sera (cf. Gallot et al (1986)).

TABLE 1
Serology scores.

| Construct used in the | | | | Construct used in the | | | |
|---|---|---|---|---|---|---|---|
| preparation of the | | | | preparation of the | | | |
| kodecytes | V | VI | I | kodecytes | V | VI | I |
| | | | | Sera | | | |
| Monoclonal preparation | Serology score | | | (Blood group) | Serology score | | |
| Anti-A Ortho-BioClone | ++++ | ++++ | 0 | O | ++++ | ++++ | 0 |
| Anti-A ortho | ++++ | ++++ | 0 | O | ++++ | ++++ | 0 |
| Anti-A ALBAclone | ++++ | ++++ | 0 | O | ++++ | ++++ | +++ |
| Anti-A Millipore | ++++ | ++++ | ++ | O | ++++ | ++++ | + |
| Anti-A Biotec | ++++ | ++++ | ++++ | O | +++ | ++++ | 0 |
| Anti-A Ortho-MoltenClone | ++++ | ++++ | ++++ | O | ++++ | ++++ | 0 |
| Anti-A (ABO1) Diagast | +++ | ++++ | 0 | O | ++++ | ++++ | 0 |
| Anti-A Diagast | ++++ | ++++ | 0 | O | +++ | ++++ | 0 |
| Anti-A Span | ++++ | ++++ | 0 | O | ++++ | ++++ | 0 |
| Anti-A CSL Epiclone | ++++ | ++++ | 0 | O | ++++ | ++++ | 0 |
| Anti-A CSL | ++++ | ++++ | 0 | O | +++ | ++++ | 0 |
| Anti-A Immucor | ++++ | ++++ | ++ | O | +++ | +++ | 0 |
| Anti-A BRG | ++++ | ++++ | 0 | O | ++++ | ++++ | ++ |
| Anti-A Diagnostics Scotland | ++++ | ++++ | 0 | O | ++++ | +++ | 0 |
| Anti-A Lorne | ++++ | ++++ | 0 | O | ++ | +++ | 0 |
| Anti-A Biotest | ++++ | ++++ | 0 | O | ++++ | +++ | 0 |
| Anti-B Dominion (Novaclone) | 0 | 0 | 0 | O | ++++ | ++++ | 0 |
| Anti-B Ortho-BioClone | 0 | 0 | 0 | O | ++ | ++++ | 0 |
| Anti-B ortho | 0 | 0 | 0 | O | +++ | ++++ | 0 |
| Anti-B ALBAclone | 0 | 0 | 0 | O | ++ | ++ | 0 |
| Anti-B Millipore | 0 | 0 | 0 | B | ++++ | ++++ | 0 |
| Anti-B Biotec | 0 | 0 | 0 | B | ++++ | ++++ | 0 |
| Anti-B Diagnostics Scotland | 0 | 0 | 0 | B | ++++ | ++++ | 0 |
| Anti-B Ortho-MoltenClone | 0 | 0 | 0 | B | ++++ | ++++ | 0 |
| Anti-B Gama | 0 | 0 | 0 | B | +++ | ++++ | 0 |
| Anti-B (ABO2) Diagast | 0 | 0 | 0 | B | +++ | ++++ | 0 |
| Anti-B Diagast | 0 | 0 | 0 | B | ++++ | ++++ | 0 |
| Anti-B Span | 0 | 0 | 0 | B | ++++ | ++++ | 0 |
| Anti-B CSL Epiclone | 0 | 0 | 0 | B | +++ | ++++ | ++ |
| Anti-B CSL | 0 | 0 | 0 | B | ++++ | +++ | 0 |
| Anti-B Immucor | 0 | 0 | 0 | B | ++ | +++ | 0 |
| Anti-A/B Immucor | ++++ | ++++ | ++ | B | ++ | ++++ | 0 |
| Anti-A/B Dominion (Novaclone) | ++++ | ++++ | 0 | B | +++ | +++ | 0 |
| Anti-A/B Ortho-BioClone | ++++ | ++++ | + | B | ++ | ++++ | 0 |
| Anti-A/B ALBAclone | ++++ | ++++ | 0 | B | +++ | +++ | 0 |
| Anti-A/B Ortho-MoltenClone | ++++ | ++++ | + | B | +++ | +++ | 0 |
| Diagast Anti-A/B (ABO3) | + | ++++ | 0 | B | +++ | +++ | 0 |
| Anti-A, B Diagast | 0 | ++++ | 0 | B | +++ | ++ | 0 |
| Anti-A/B CSL Epiclone | ++++ | ++++ | 0 | B | 0 | +++ | 0 |
| Anti-A, B CSL | ++++ | ++++ | 0 | B | +++ | +++ | 0 |
| Anti-A, B Diagnostics Scotland | ++++ | ++++ | 0 | A | 0 | + | 0 |
| | | | | A | 0 | ++ | 0 |
| | | | | A | 0 | 0 | 0 |
| | | | | A | 0 | 0 | 0 |
| | | | | A | 0 | ++ | 0 |
| | | | | A | ++ | ++ | 0 |
| | | | | A | 0 | +++ | 0 |
| | | | | A | 0 | 0 | 0 |
| | | | | A | 0 | 0 | 0 |
| | | | | A | 0 | 0 | 0 |
| | | | | A | 0 | 0 | 0 |
| | | | | A | ++ | 0 | 0 |
| | | | | A | 0 | 0 | 0 |
| | | | | A | 0 | 0 | 0 |
| | | | | A | 0 | 0 | 0 |
| | | | | A | 0 | 0 | 0 |
| | | | | A | 0 | ++ | 0 |
| | | | | A | 0 | +++ | 0 |
| | | | | A | 0 | 0 | 0 |
| | | | | A | ++ | +++ | 0 |

Although the invention has been described with reference to embodiments or examples it should be appreciated that variations and modifications may be made to these embodiments or examples without departing from the scope of the invention. Where known equivalents exist to specific elements, features or integers, such equivalents are incorporated as if specifically referred to in this specification. In particular, variations and modifications to the embodiments or examples that include elements, features or integers disclosed in and selected from the referenced publications are within the scope of the invention unless specifically disclaimed. The advantages provided by the invention and discussed in the description may be provided in the alternative or in combination in these different embodiments of the invention.

### REFERENCES

Alvarez et al (1995) Aminoborohydrides. 8. A facile reduction of aliphatic and benzylic azides to the corresponding amines in high yield and purity using lithium, N,N-Dimethylaminobrorhydride Tetrahedron Letters, 36(15), 2567-2570.
Barragan et al (2001) A mannose-6-phosphonate-cholesterylamine conjugate as a specific molecular adhesive linking cancer cells with vesicles Chem. Commun. 85-86.
Beigelman et al (1999) Novel compositions for the delivery of negatively charged molecules Publ. no. WO 99/05094.
Boonyarattanakalin et al (2006) Synthesis of an artificial cell surface receptor that enables oligohistidine affinity tags to function as metal-dependent cell-penetrating peptides J. Am. Chem. Soc. 128, 386-387.
Booth et al (2001) Synthesis of novel biotin anchors Tetrahedron, 57(49), 9859-9866.
Bovin et al (2005) Synthetic membrane anchors Publication no. WO 2005/090368.
Bovin et al (2009) Functional lipid constructs Publication no. WO 2009/048343.
Carrion et al (2001) Preparation of long-circulating immunoliposomes using PEG-cholesterol conjugates: effect of the spacer arm between PEG and cholesterol on liposomal characteristics Chemistry and Physics of Lipids, 113(1-2), 97-110.
Düffels et al (2000) Synthesis of high-mannose type neoglycolipids: Active targeting of liposomes to macrophages in gene therapy Chem. Eur. J. 6(8) 1416-1430.
Gallot et al (1986) Study by X-ray diffraction of the geometrical shape of glycoprotein sugar chains in two model glycoconjugates, a liposaccharide and phospholiposaccharide, having the same sugar chain Carbohydrate Research, 149, 309-318.
Herscovici et al (2002) Polythiourea lipid derivatives Publ. no. WO 02/092558.
Knölker et al (2008) Cholesterylamines for the treatment and prevention of infectious diseases Publ. no. WO 2008/068037.
Korchagina et al (2008) Fluorescent cell markers Publication no. WO 2008/030115.
Lingwood et al (2011) Cholesterol modulates glycolipid conformation and receptor activity Nature Chemical Biology, 7, 260-262.
Orr et al (1979) Synthetic glycolipids and the lectin-mediated aggregation of liposomes The Journal of Biological Chemistry, Vol 254, No. 11, Issue of June 10, 4721-4725.
Pacuszka et al (1991) Neoglycolipid analogues of ganglioside GM1 as functional receptors of cholera toxin Biochemistry, 30(10), 2563-2570.
Peterson (2006) Synthetic mimics of mammalian cell surface receptors: Methods and compositions Publ. no. US 2006/0229235.
Reddy et al (1999) A novel, simple, chemoselective and practical protocol for the reduction of azides using In/NH₄Cl Tetrahedron Letters 40, 3937-3938.
Reddy et al (2000) A new novel and practical one pot methodology for conversion of alcohols to amines Synthetic Communications, 30(12), 2233-2237.
Spedden (2008) Biomimetic particles and films for pathogen capture and other uses Publ. No. WO 2008/154603.
Spencer et al (2004) Benzophenone-containing cholesterol surrogates: synthesis and biological evaluation Journal of Lipid Research, 45, 1510-1518.
Vidal et al (2003) A convenient synthetic route to mannose 6-phosphonate-cholesteryl conjugate Heteroatom Chemistry, Vol. 14, Number 3, 241.
Weinberg et al (2009) Peptide-lipid constructs and their use in diagnostic and therapeutic applications Publication no. WO 2009/035347.
Zurbriggen et al (2006) Lyophilization of virosomes Publication no. WO 2006/069719.

1-38. (canceled)

39. A method of localizing a hydrophilic biofunctional moiety (F) to a surface comprising the step of contacting the surface with an aqueous solution of a water dispersible construct of the structure F-S-S′ where F is the biofunctional moiety, S is a spacer covalently linking F to S′, and S′ is steryl.

40. The method of claim 39 where the biofunctional moiety (F) is selected from the group consisting of: biotin and O-linked oligosaccharides.

41. The method of claim 40 where the biofunctional moiety (F) is an oligosaccharide selected from the group consisting of: GalNAcα3 (Fucα2) Galβ-; Galα3 (Fucα2) Galβ-; GalNα3 (Fucα2) Galβ-; Fucα2Galβ-; Galβ4GlcNAcβ3 (Galβ4GlcNAcβ6) Galβ-; Galβ4GlcNAcβ3-; Galβ4Glcβ-; Galβ3GlcNAcβ-; Galβ3 (Fucα4) GlcNAcβ-; Fucα2Galβ3(Fucα4)GlcNAcβ-; GalNAcα3 (Fucα2)Galβ3 (Fucα4) GlcNAcβ-; Galα3 (Fucα2) Cairn (Fucα4) GlcNAcβ-; Galβ4 (Fucα3) GlcNAcβ-; Fucα2Galβ4 (Fucα3) GlcNAcβ-; NeuAca2-3Galβ3 (Fucα4) GlcNAcβ-; NeuAcα2-3Galβ4 (Fucα3) GlcNAcβ-; GalNAcβ4 (NeuAcα2-3) Galβ4-; Galβ3GalNAcα-; NeuAcα2-3Galβ4-; NeuAcα2-6Gal8β-; Galα4Galβ4-; GalNAcβ3Galα4Galβ4-; Galα4Galβ4GlcNAcβ3-; Galβ3GalNAcβ3Galα4-; NeuAcα2-3Galβ3GalNAcβ3Galα4-; Galα3Galβ-; GalNAcα3GalNAcβ3Galα4-; GalNAcβ3GalNAcβ3Galα4-; Galβ1-4GlcNAc; Galβ1-3GlcNAc; SAα2-6Galβ1-4Glc; SAα2-3Galβ1-4Glc; SAα2-6Galβ1-4GlcNAc; SAα2-3Galβ1-4GlcNAc; SAα2-3Galβ1-3GlcNAc; Galβ1-4 (Fucα1-3) GlcNAc; Galβ1-3 (Fucα1-3) GlcNAc; SAα2-3Galβ1-3 (Fucα1-4) GlcNAc; SAα2-3Galβ1-4 (Fucα1-3) GlcNAc; Galβ1-4GlcNAcβ1-4GlcNAc; Galβ1-3GlcNAcβ1-4GlcNAc; SAα2-6Galβ1-4GlcNAcβ1-4GlcNAc; SAα2-3Galβ1-4GlcNAcβ1-4GlcNAc; SAα2-3Galβ1-3GlcNAcβ1-4GlcNAc; Galβ1-4 (Fucα1-3) GlcNAcβ1-4GlcNAc; Galβ1-3 (Fucα1-4) GlcNAcβ1-4GlcNAc; SAα2-3Galβ1-3 (Fucα1-4) GlcNAcβ1-4GlcNAc; SAα2-3Galβ1-4(Fucα1-3) GlcNAcβ1-4GlcNAc; SAα2-3Galβ1-3 (Fucα1-4) GlcNAcβ1-4Gal; SAα2-3Galβ1-4 (Fucα1-3) GlcNAcβ1-4Gal; SAα2-3Galβ1-4GlcNAcβ1-3Galβ1-4 (Fucα1-3) GlcNAc; SAα2-6Galβ1-4GlcNAcβ1-3Galβ1-4 (Fucα1-3) GlcNAc; SAα2-3Galβ1-4 (Fucα1-3) GlcNAcβ1-3Galβ1-4Glc; SAα2-6Galβ1-4 (Fucα1-3) GlcNAcβ1-3Galβ1-4Glc; SAα2-3Galβ1-4GlcNAcβ1-3Galβ1-4 (Fucα1-3) GlcNAcβ1-3Galβ1-4Glc; SAα2-6Galβ1-4GlcNAcβ1-3Galβ1-4 (Fucα1-3) GlcNAcβ1-3Galβ1-4Glc; SAα2-3Galβ1-4(Fucα1-3) GlcNAcβ1-3Galβ1-4 (Fucα1-3) GlcNAcβ1-3Galβ1-4Glc; SAα2-6Galβ1-4 (Fucα1-3) GlcNAcβ1-3Galβ1-4 (Fucα1-3) GlcNAcβ1-3Galβ1-4Glc; SAα2-3Galβ1-4 (Fucα1-3) GlcNAcβ1-3Galβ1-4(Fucα1-3) Glc; SAα2-6Galβ1-4 (Fucα1-3) GlcNAcβ1-3Galβ1-4 (Fucα1-3) Glc; SAα2-3Galβ1-4GlcNAcβ1-3Galβ1-4 (Fucα1-3) GlcNAcβ1-3Galβ1-4 (Fucα1-3) Glc; SAα2-6Galβ1-4GlcNAcβ1-3Galβ1-4 (Fucα1-3) GlcNAcβ1-3Galβ1-4 (Fucα1-3) Glc; SAα2-3Galβ1-4 (Fucα1-3) GlcNAcβ1-3Galβ1-4 (Fucα1-3) GlcNAcβ1-3Galβ1-4 (Fucα1-3) Glc; SAα2-6Galβ1-4 (Fucα1-3) GlcNAcβ1-3Galβ1-4 (Fucα1-3) GlcNAcβ1-3Galβ1-4 (Fucα1-3) Glc; SAα2-3Galβ1-3 (Fucα1-4) GlcNAc; SAα2-6Galβ1-3 (Fucα1-4) (GlcNAc; SAα2-3Glaβ1-3GlcNAcβ1-4Galβ1-4 (Fucα1-3) GlcNAc; SAα2-6Galβ1-3GlcNAcβ1-4Galβ1-4 (Fucα1-3) GlcNAc; SAα2-3Galβ1-3 (Fucα1-4) GlcNAcβ1-3Galβ1-4 (Fucα1-3) GlcNAc; SAα2-6Galβ1-3 (Fucα1-4) GlcNAcβ1-3Galβ1-4 (Fucα1-3) GlcNAc; SAα2-3Galβ1-3 (Fucα1-4) GlcNAcβ1-3Galβ1-4Glc; SAα2-6Galβ1-3 (Fucα1-4) GlcNAcβ1-3Galβ1-4Glc; SAα2-3Glaβ1-3GlcNAcβ1-4Galβ1-4 (Fucα1-3) GlcNAcβ1-3Galβ1-4Glc; SAα2-6Galβ1-3GlcNAcβ1-4Galβ1-4(Fucα1-3) GlcNAcβ1-3Galβ1-4Glc; SAβ2-3Glaβ1-3(Fucα1-4)GlcNAcβ1-3Galβ1-4(Fucα1-3) GlcNAcβ1-3Galβ1-4Glc; SAα2-6Galβ1-3(Fucα1-4) GlcNAcβ1-3Galβ1-4 (Fucα1-3) GlcNAcβ1-3Galβ1-4Glc; SAα2-3Galβ1-3 (Fucα1-4) GlcNAcβ1-3Galβ1-4 (Fucα1-3) Glc; SAα2-6Galβ1-3 (Fucα1-4) GlcNAcβ1-3Galβ1-4 (Fucα1-3) Glc; SAα2-3Galβ1-3GlcNAcβ1-4Galβ1-4 (Fucα1-3) GlcNAcβ1-3Galβ1-4 (Fucα1-3) Glc; SAα2-6Galβ1-3GlcNAcβ1-4Galβ1-4 (Fucα1-3) GlcNAcβ1-3Galβ1-4 (Fucα1-3) Glc; SAα2-3Galβ1-3 (Fucα1-4) GlcNAcβ1-3Galβ1-4(Fucα1-3) GlcNAcβ1-3Galβ1-4 (Fucα1-3) Glc; SAα2-6Galβ1-3 (Fucα1-4) GlcNAcβ1-3Galβ1-4(Fucα1-3) GlcNAcβ1-3Galβ1-4 (Fucα1-3) Glc; SAα2-3Galβ1-4GlcNAcβ1-3Glaβ1-3 (Fucα1-4) GlcNAc; SAα2-6Galβ1-4GlcNAcβ1-3Glaβ1-3 (Fucα1-4) GlcNAc; SAα2-3Galβ1-4 (Fucα1-3) GlcNAcβ1-3Galβ1-3 (Fucα1-4) GlcNAc; SAα2-6Galβ1-4 (Fucα1-3) GlcNAcβ1-3Glaβ1-3 (Fucα1-4) GlcNAc; SAα2-3Galβ1-4GlcNAcβ1-3Galβ1-3 (Fucα1-4) GlcNAcβ1-3Galβ1-4Glc; SAα2-6Galβ1-4GlcNAcβ1-3Galβ1-3 (Fucα1-4) GlcNAcβ1-3Galβ1-4Glc; SAα2-3Galβ1-4 (Fucα1-3) GlcNAcβ1-3Galβ1-3 (Fucα1-4) GlcNAcβ1-3Galβ1-4Glc; SAα2-6Galβ1-4 (Fucα1-3) GlcNAcβ1-3Galβ1-3(Fucα1-4) GlcNAcβ1-3Galβ1-4Glc; SAα2-3Galβ1-4 (Fucα1-3) GlcNAcβ1-3Galβ1-3 (Fucα1-4) Glc; SAα2-6Galβ1-4 (Fucα1-3) GlcNAcβ1-3Galβ1-3 (Fucα1-4) Glc; SAα2-3Galβ1-4GlcNAcβ1-3Glaβ1-3 (Fucα1-4) GlcNAcβ1-3Galβ1-4 (Fucα1-3) Glc; SAα2-6Galβ1-4GlcNAcβ1-3Glaβ1-3 (Fucα1-4) GlcNAcβ1-3Galβ1-4 (Fucα1-3) Glc; SAα2-3Galβ1-4 (Fucα1-3) GlcNAcβ1-3Glaβ1-3 (Fucα1-4) GlcNAcβ1-3Galβ1-4 (Fucα1-3) Glc; and SAα2-6Galβ1-4 (Fucα1-3) GlcNAcβ1-3Glaβ1-3 (Fucα1-4) GlcNAcβ1-3Galβ1-4 (Fucα1-3) Glc, where SA is sialic acid.

42. The method of claim 39 where S′ is selected from the group consisting of: campesteryl, cholesteryl, ergosteryl, sitosteryl and stigmasteryl.

43. The method of claim 42 where S′ is cholesteryl.

44. The method of claim 39 where the water dispersible construct is of the structure: 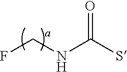
and a is the integer 2, 3 or 4.

45. The method of claim 39 where the water dispersible construct is of the structure: 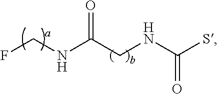
a is the integer 2, 3 or 4 and b is the integer 3, 4 or 5.

46. The method of claim 39 where the water dispersible construct is of the structure: 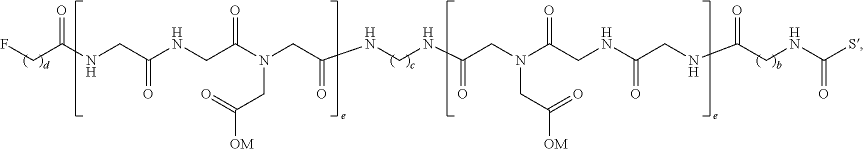
where b is the integer 3, 4 or 5, c is the integer 1, 2 or 3, d is the integer 3, 4 or 5, e is the integer 1, 2 or 3 and M is a monovalent cation.

47. The method of claim 44 where the water dispersible construct is of the structure: 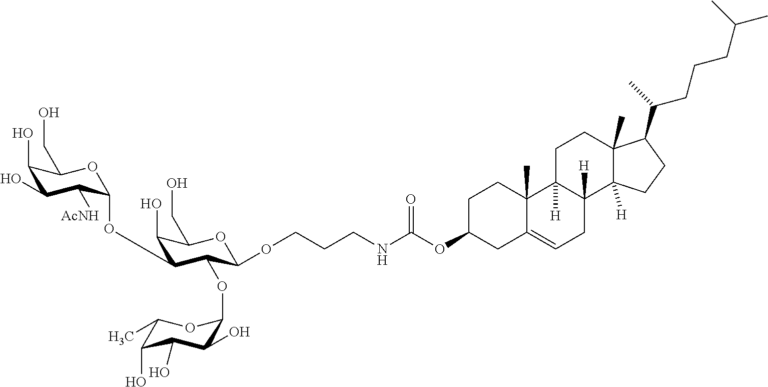
designated Aₜᵣᵢ-S₁-chol (I).

48. The method of claim 45 where the water dispersible construct is of the structure: 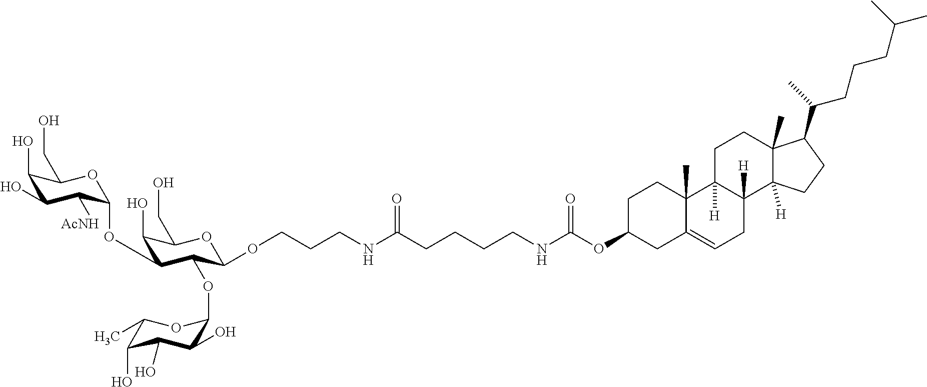
designated Aₜᵣᵢ-S₁-S₂-chol (II).

49. The method of claim 46 where the water dispersible construct is of the structure: 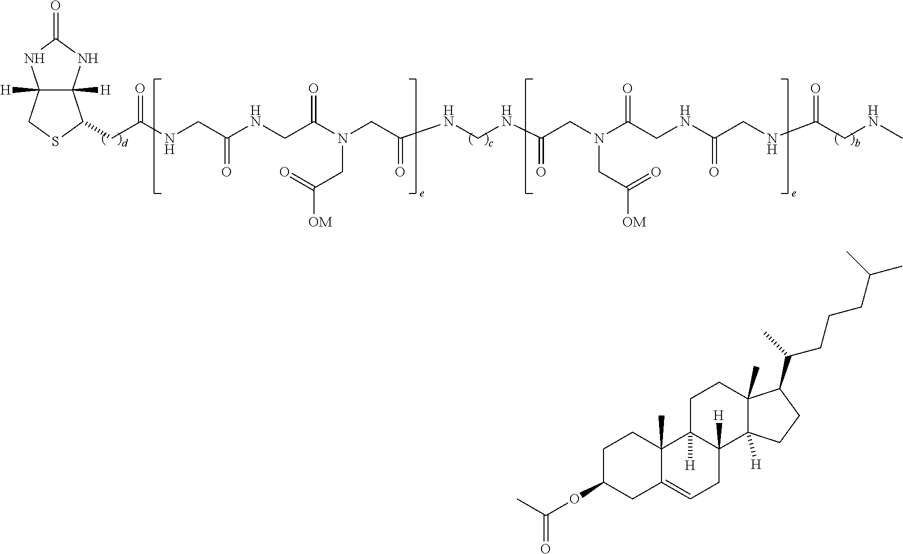
designated Biotin-S₃-S₄-S₂-chol (III).

50. The method of claim 39 where the surface is the membrane of a cell.

51. The method of claim 39 where the surface is the membrane of an enveloped virus.

52. The method of claim 39 where the surface is the lipid bilayer of a liposome or a virosome.

53. The method of claim 39 where the step of contacting the surface with an aqueous solution of a water dispersible construct is by propelling droplets of the aqueous solution from a plurality of orifices located in a monolithic print head of an inkjet printer onto at least one discrete area on the surface.

54. The method of claim 53 where the volume of each of the droplets is 1 to 100 picolitres (pL).

55. The method of claim 54 where the volume of each of the droplets is 1 to 50 μL.

56. The method of claim 55 where the volume of each of the droplets is 1 to 5 μL.

57. The method of claim 53 where the concentration of the water dispersible construct in the aqueous solution is 1 μmolar (μM) to 10 mmolar (mM).

58. The method of claim 57 where the concentration of the construct in the aqueous solution is 10 μM to 10 mM.

59. The method of claim 58 where the concentration of the construct in the aqueous solution is 0.1 to 10 mM.

60. The method of claim 53 where the at least one discrete area is in the shape of a symbol readable by optical character recognition (OCR) apparatus.

61. The method of claim 60 where the at least one discrete area is in the shape of a symbol comprising one or more alphanumeric characters.

62. The method of claim 53 where the at least one discrete area is a pattern comprising a combination of indicia to which the aqueous solution is applied at different densities (amount per unit area).
